(19) United States
(12) Patent Application Publication
(10) Pub. No.: US 2004/0248286 A1
(43) Pub. Date: Dec. 9, 2004

(54) Nucleic acid molecules that are differentially regulated in a bipolar disorder and uses thereof

(76) Inventors: Christine Konradi, Chestnut Hill, MA (US); Stephan Heckers, Chestnut Hill, MA (US)

Correspondence Address: CLARK & ELBING LLP, 101 FEDERAL STREET, BOSTON, MA, 02110, US

(21) Appl. No.: 10/804,950

(22) Filed: Mar. 19, 2004

Related U.S. Application Data
(60) Provisional application No. 60/456,873, filed on Mar. 21, 2003.
(60) Provisional application No. 60/516,527, filed on Oct. 30, 2003.

(51) Int. Cl. C12Q 1/68; C07H 21/04; C12M 1/34

(52) U.S. Cl. 435/287.2; 435/6; 536/23.2

(57) ABSTRACT

In general, this invention relates to nucleic acid and amino acid sequences that are differentially regulated in a bipolar disorder, and the use of these sequences as targets for the development of compounds that treat a bipolar disorder. In addition, the methods and sequences described herein are useful for the diagnosis, monitoring, and treatment of a bipolar disorder in a human patient.

### CROSS REFERENCE TO RELATED APPLICATIONS

This application claims the benefit of U.S. provisional patent applications 60/456,873 and 60/516,527, filed Mar. 21, 2003 and Oct. 30, 2003, respectively, each of which is incorporated by reference.

### BACKGROUND OF THE INVENTION

The invention features methods and compositions for the diagnosis, monitoring, and treatment of bipolar disorder in human patients, as well as methods and compositions for the identification of new candidate therapeutics.

Bipolar disorder is a mood disorder characterized by pathological mood swings from mania to depression. The disorder, which typically appears during adolescence or early adulthood, affects approximately 2.3 million American adults, or about 1.2% of the U.S. population. While bipolar disorder shows a high degree of heritability, and several studies have reported linkage of bipolar disorder to chromosomal loci, no single locus has been repeatedly linked to bipolar disorder and the etiology and disease mechanisms remain unknown.

Current treatments for bipolar disorder include mood stabilizing drugs, such as lithium and carbamazepine, and anticonvulsant medications, such as valproate, lamotrigine, gabapentin, and topiramate. For some patients with refractory bipolar symptoms, atypical antipsychotic drugs, such as clozapine or olanzapine, may be helpful. In severe cases, electroconvulsive therapy is used to treat severe depression and/or mania. Despite the variety of available medications, many patients treated for bipolar disorders continue to experience symptoms. Moreover, many patients discontinue their medication due to adverse side effects. Improved therapeutics with fewer side-effects are needed for the treatment of bipolar disorders. Additionally, current methods for diagnosing bipolar disorders, which rely on patient-reporting and clinical observation, could be improved if more accurate and efficient diagnostic methods were available.

### SUMMARY OF THE INVENTION

The invention features methods and compositions for the diagnosis, monitoring, and treatment of bipolar disorder in human patients, as well as methods and compositions for the identification of new candidate therapeutics.

In one aspect, the invention generally features a microarray comprising at least two, five, ten, fifteen, twenty, thirty, forty, fifty, or sixty nuclear encoded mitochondrial energy metabolism nucleic acid molecules, or fragments thereof, bound to a solid support, where at least 90%, 95%, or 100% of the nucleic acid molecules on the support are nuclear encoded mitochondrial energy metabolism nucleic acid molecules.

In another aspect, the invention features a microarray comprising at least two, five, ten, fifteen, twenty, thirty, forty, fifty, or sixty nuclear encoded mitochondrial energy metabolism polypeptides, or fragments thereof, bound to a solid support where at least 90%, 95%, or 100% of the polypeptides on the support are nuclear encoded mitochondrial energy metabolism polypeptides.

In another aspect, the invention provides a method of diagnosing a patient as having, or having a propensity to develop, a bipolar disorder. The method involves determining the level of expression of a nuclear encoded mitochondrial energy metabolism nucleic acid molecule in a patient sample, where a decreased level of expression relative to the level of expression in a control sample, indicates that the patient has or has a propensity to develop a bipolar disorder. In one preferred embodiment, the patient sample is a blood sample. In other preferred embodiments, the level of expression is determined using a microarray or quantitative real-time PCR.

In a related aspect, the invention provides a method of diagnosing a patient having, or having a propensity to develop, a bipolar disorder. The method involves determining the level of expression of a nuclear encoded mitochondrial energy metabolism polypeptide in a patient sample, where a decreased level of expression relative to the level of expression in a control sample, indicates that the patient has or has a propensity to develop a bipolar disorder. In one preferred embodiment, the patient sample is a blood sample. In another embodiment, the level of expression is determined using a microarray. In preferred embodiments, the level of expression is determined in an immunological or enzymatic assay.

In another aspect, the invention provides a method of monitoring a patient having a bipolar disorder. The method involves determining the level of expression of a nuclear encoded mitochondrial energy metabolism nucleic acid or polypeptide encoded by the nucleic acid molecule in a patient sample, where an alteration in the level of expression relative to the level of expression in a control sample indicates the severity of bipolar disorder in the patient. In one embodiment, the control sample is a normal patient sample or a reference sample taken from the patient. In one preferred embodiment, the patient sample is a blood sample. In another preferred embodiment, the patient is being treated for a bipolar disorder.

In another aspect, the invention features a diagnostic kit for the diagnosis of a bipolar disorder in a patient comprising a nucleic acid sequence, or fragment thereof, that encodes a polypeptide selected from the group consisting of an ATP synthase (mitochondrial F0 complex, subunit c, isoform 3), VDAC1 pseudogene (porin protein, isoform 1), ubiquinone-binding protein, ATP synthase (mitochondrial F0 complex, subunit d), mitochondrial ribosomal protein L3, cytochrome c oxidase subunit VIIb, ATP synthase (mitochondrial F0 complex, subunit f, isoform 2), dynamin 1-like protein, voltage-dependent anion channel 2 (porin), Cytochrome c oxidase subunit VIIa polypeptide 2 (liver), ATP synthase (mitochondrial F1 complex, O subunit), voltage-dependent anion channel 1 (porin), single-stranded DNA binding protein, fumarate hydratase, solute carrier family 25 (member 4), ATP synthase (mitochondrial F1 complex gamma polypeptide 1), NADH dehydrogenase ((ubiquinone) 1 alpha/beta subcomplex 1, 8 kDa), and 3-oxoacid CoA transferase.

In a related aspect, the invention provides a method of treating or preventing a bipolar disorder. The method involves administering to a patient in need of such treatment an effective amount of a pharmaceutical composition comprising a nucleic acid molecule selected from the group consisting of an ATP synthase (mitochondrial F0 complex, subunit c, isoform 3), VDAC1 pseudogene (porin protein, isoform 1), ubiquinone-binding protein, ATP synthase (mitochondrial F0 complex, subunit d), mitochondrial ribosomal protein L3, cytochrome c oxidase subunit VIIb, ATP synthase (mitochondrial F0 complex, subunit f, isoform 2), dynamin 1-like protein, voltage-dependent anion channel 2 (porin), Cytochrome c oxidase subunit VIIa polypeptide 2 (liver), ATP synthase (mitochondrial F1 complex, O subunit), voltage-dependent anion channel 1 (porin), single-stranded DNA binding protein, fumarate hydratase, solute carrier family 25 (member 4), ATP synthase (mitochondrial F1 complex gamma polypeptide 1), NADH dehydrogenase ((ubiquinone) 1 alpha/beta subcomplex 1, 8 kDa), and 3-oxoacid CoA transferase.

In another related aspect, the invention provides a method of treating or preventing a bipolar disorder. The method involves administering to a patient in need of such treatment an effective amount of a pharmaceutical composition that increases expression of a nuclear encoded mitochondrial energy metabolism polypeptide.

In another aspect, the invention provides another method of identifying a candidate compound that ameliorates a bipolar disorder. This method involves contacting a cell that expresses a nuclear encoded mitochondrial energy metabolism nucleic acid molecule with a candidate compound, and comparing the level of expression of the nucleic acid molecule in the cell contacted by the candidate compound with the level of expression in a control cell not contacted by the candidate compound, where an increase in expression of the nuclear encoded mitochondrial energy metabolism nucleic acid molecule identifies the candidate compound as a candidate compound that ameliorates a bipolar disorder. In preferred embodiments, the increase in expression is an increase in transcription or an increase in translation.

In another aspect, the invention provides a method of treating or preventing a bipolar disorder. The method involves administering to a patient in need of such treatment an effective amount of a pharmaceutical composition comprising a nuclear encoded mitochondrial energy metabolism polypeptide selected from the group consisting of an ATP synthase (mitochondrial F0 complex, subunit c, isoform 3), VDAC1 pseudogene (porin protein, isoform 1), ubiquinone-binding protein, ATP synthase (mitochondrial F0 complex, subunit d), mitochondrial ribosomal protein L3, cytochrome c oxidase subunit VIIb, ATP synthase (mitochondrial F0 complex, subunit f, isoform 2), dynamin 1-like protein, voltage-dependent anion channel 2 (porin), Cytochrome c oxidase subunit VIIa polypeptide 2 (liver), ATP synthase (mitochondrial F1 complex, O subunit), voltage-dependent anion channel 1 (porin), single-stranded DNA binding protein, fumarate hydratase, solute carrier family 25 (member 4), ATP synthase (mitochondrial F1 complex gamma polypeptide 1), NADH dehydrogenase ((ubiquinone) 1 alpha/beta subcomplex 1, 8 kDa), and 3-oxoacid CoA transferase, formulated in a pharmaceutically acceptable carrier.

In another aspect, the invention provides a method of treating or preventing a bipolar disorder. The method involves administering to a patient in need of such treatment an effective amount of a pharmaceutical composition comprising a nuclear encoded mitochondrial energy metabolism nucleic acid molecule selected from the group consisting of an ATP synthase (mitochondrial F0 complex, subunit c, isoform 3), VDAC1 pseudogene (porin protein, isoform 1), ubiquinone-binding protein, ATP synthase (mitochondrial F0 complex, subunit d), mitochondrial ribosomal protein L3, cytochrome c oxidase subunit VIIb, ATP synthase (mitochondrial F0 complex, subunit f, isoform 2), dynamin 1-like protein, voltage-dependent anion channel 2 (porin), Cytochrome c oxidase subunit VIIa polypeptide 2 (liver), ATP synthase (mitochondrial F1 complex, O subunit), voltage-dependent anion channel 1 (porin), single-stranded DNA binding protein, fumarate hydratase, solute carrier family 25 (member 4), ATP synthase (mitochondrial F1 complex gamma polypeptide 1), NADH dehydrogenase ((ubiquinone) 1 alpha/beta subcomplex 1, 8 kDa), and 3-oxoacid CoA transferase, formulated in a pharmaceutically acceptable carrier.

In a related aspect, the invention provides another method of identifying a candidate compound that ameliorates a bipolar disorder. This method involves contacting a cell that expresses a nuclear encoded mitochondrial energy metabolism polypeptide with a candidate compound, and comparing the level of expression of the polypeptide in the cell contacted,by the candidate compound with the level of polypeptide expression in a control cell not contacted by the candidate compound, where an increase in the expression of the nuclear encoded mitochondrial energy metabolism polypeptide identifies the candidate compound as a candidate compound that ameliorates a bipolar disorder. In preferred embodiments, the increase in expression is assayed using an immunological assay, an enzymatic assay, or a radioimmunoassay.

In another aspect, the invention features a further method of identifying a candidate compound that ameliorates a bipolar disorder. This method involves contacting a cell that expresses a nuclear encoded mitochondrial energy metabolism polypeptide with a candidate compound, and comparing the biological activity of the polypeptide in the cell contacted by the candidate compound with the level of biological activity in a control cell not contacted by the candidate compound, where an increase in the biological activity of the nuclear encoded mitochondrial energy metabolism polypeptide identifies the candidate compound as a candidate compound that ameliorates a bipolar disorder. In preferred embodiments, the increase in expression is assayed using an immunological assay, an enzymatic assay, or a radioimmunoassay.

In a related aspect, the invention provides yet another method of identifying a candidate compound that ameliorates a bipolar disorder. This method involves a) contacting a cell with a candidate compound; b) obtaining a nucleic acid from the cell; c) contacting a microarray described above with the nucleic acid; and d) detecting an increase in expression level of a nuclear encoded mitochondrial energy metabolism nucleic acid molecule in the cell contacted with the candidate compound compared to a control cell, where the increase identifies the candidate compound as a candidate compound that ameliorates a bipolar disorder.

In a related aspect the invention provides A method of identifying a candidate compound that ameliorates a bipolar disorder. The method involves a) contacting a microarray of a previous aspect with a candidate compound; and b) detecting binding of the candidate compound to a nuclear encoded mitochondrial energy metabolism polypeptide, where the binding identifies the compound as a candidate compound that ameliorates a bipolar disorder.

In another aspect, the invention features a collection of primer sets, each of the primer sets comprising at least two, five, ten, fifteen, twenty, thirty, forty, fifty, or sixty primers that bind to a nuclear encoded mitochondrial energy metabolism nucleic acid molecule that encodes a polypeptide selected from the group consisting of an ATP synthase (mitochondrial F0 complex, subunit c, isoform 3), VDAC1 pseudogene (porin protein, isoform 1), ubiquinone-binding protein, ATP synthase (mitochondrial F0 complex, subunit d), mitochondrial ribosomal protein L3, cytochrome c oxidase subunit VIIb, ATP synthase (mitochondrial F0 complex, subunit f, isoform 2), dynamin 1-like protein, voltage-dependent anion channel 2 (porin), Cytocbrome c oxidase subunit VIIa polypeptide 2 (liver), ATP synthase (mitochondrial F1 complex, O subunit), voltage-dependent anion channel 1 (porin), single-stranded DNA binding protein, fumarate hydratase, solute carrier family 25 (member 4), ATP synthase (mitochondrial F1 complex gamma polypeptide 1), NADH dehydrogenase ((ubiquinone) 1 alpha/beta subcomplex 1, 8 kDa), and 3-oxoacid CoA transferase under high stringency conditions, the collection comprising at least two, five, ten, twenty, forty, sixty, or eighty primer sets.

In another aspect, the invention features a purified nucleic acid library comprising nuclear encoded mitochondrial metabolism nucleic acid molecules, where at least 90%, 95%, or 100% of the nucleic acid molecules are nuclear encoded mitochondrial metabolism nucleic acid molecules. In preferred embodiments, the nucleic acid molecules in the library are carried in a vector. In other preferred embodiments, each of the nucleic acid molecules is fused to a reporter gene.

In another aspect, the invention provides a method of identifying a candidate compound that ameliorates a bipolar disorder. The method involves a) contacting a cell comprising one member of the library of the previous aspect; and b) measuring the expression of the reporter gene; and c) comparing the level of reporter gene expression in the cell contacted with the candidate compound with a control cell not contacted with the candidate compound, where an increase in the level of the reporter gene expression identifies the candidate compound as a candidate compound that ameliorates a bipolar disorder.

In preferred embodiments of any of the previous aspects, the nuclear encoded mitochondrial energy metabolism nucleic acid or polypeptide is a nucleic acid or polypeptide selected from the group consisting of any one or all of the following: ATP synthase (mitochondrial F0 complex, subunit c, isoform 3), VDAC1 pseudogene (porin protein, isoform 1), ubiquinone-binding protein, ATP synthase (mitochondrial F0 complex, subunit d), mitochondrial ribosomal protein L3, cytochrome c oxidase subunit VIIb, ATP synthase (mitochondrial F0 complex, subunit f, isoform 2), dynamin 1-like protein, voltage-dependent anion channel 2 (porin), Cytochrome c oxidase subunit VIIa polypeptide 2 (liver), ATP synthase (mitochondrial F1 complex, O subunit), voltage-dependent anion channel 1 (porin), single-stranded DNA binding protein, fumarate hydratase, solute carrier family 25 (member 4), ATP synthase (mitochondrial F1 complex gamma polypeptide 1), NADH dehydrogenase ((ubiquinone) 1 alpha/beta subcomplex 1, 8 kDa), and 3-oxoacid CoA transferase.

In another aspect, the invention features a microarray comprising at least two, five, ten, fifteen, or twenty proteasomal nucleic acid molecules, or fragments thereof, bound to a solid support, where at least 90%, 95%, or 100% of the nucleic acid molecules on the support are proteasomal nucleic acid molecules.

In a related aspect, the invention features a microarray comprising at least two, five, ten, fifteen, or twenty proteasomal polypeptides, or fragments thereof, bound to a solid support, where at least 90%, 95%, or 100% of the polypeptides are proteasomal polypeptides.

In another aspect, the invention provides a method of diagnosing a patient having, or having a propensity to develop, a bipolar disorder. The method involves determining the level of expression of a nucleic acid that encodes a proteasomal polypeptide in a patient sample, where a decreased level of expression relative to the level of expression in a control sample, indicates that the patient has or has a propensity to develop a bipolar disorder. In one preferred embodiment, patient sample is a blood sample. In another preferred embodiment, the level of expression is determined using a microarray.

In another aspect, the invention provides a method of diagnosing a patient having, or having a propensity to develop, a bipolar disorder. The method involves determining the level of expression of a proteasomal polypeptide in a patient sample, where a decreased level of expression relative to the level of expression in a control sample, indicates that the patient has or has a propensity to develop a bipolar disorder. In one preferred embodiment, patient sample is a blood sample. In another preferred embodiment, level of expression is determined using a microarray. In another preferred embodiment, the level of expression is determined in an immunological or enzymatic assay.

In another aspect, the invention provides a method of monitoring a patient having a bipolar disorder. The method involves determining the level of expression of a proteasomal nucleic acid molecule or polypeptide in a patient sample, where an alteration in the level of expression relative to the level of expression in a control sample indicates the severity of a bipolar disorder in the patient. In preferred embodiments, the control sample is a normal patient sample or a reference sample taken from the patient. In one preferred embodiment, the patient sample is a blood sample. In another preferred embodiment, the patient is being treated for a bipolar disorder. In other preferred embodiments, the level of expression is determined using a microarray or quantitative real-time PCR.

In another aspect, the invention features a diagnostic kit for the detection of a bipolar disorder comprising a nucleic acid molecule, or fragment thereof, that encodes a polypeptide selected from the group consisting of a Sec61 gamma, protein-L-isoaspartate (D-aspartate) O-methyltransferase, F-box only protein 9, and proteasome subunit z, where the kit diagnoses a bipolar disorder.

In another aspect, the invention provides a method of treating or preventing a bipolar disorder. The method involves administering to a patient in need of such treatment an effective amount of a pharmaceutical composition comprising a nuclear encoded mitochondrial energy metabolism polypeptide selected from the group consisting of a Sec61 gamma, protein-L-isoaspartate (D-aspartate) O-methyltransferase, F-box only protein 9, and proteasome subunit z.

In a related aspect, the invention features a method of treating or preventing a bipolar disorder. The method involves administering to a patient in need of such treatment an effective amount of a pharmaceutical composition comprising a nucleic acid molecule encoding a nuclear encoded mitochondrial energy metabolism polypeptide selected from the group consisting of a Sec6l gamma, protein-L-isoaspartate (D-aspartate) O-methyltransferase, F-box only protein 9, and proteasome subunit z.

In another related aspect, the invention features a-method of treating or preventing a bipolar disorder. The method involves administering to a patient in need of such treatment an effective amount of a pharmaceutical composition that increases expression of a proteasomal polypeptide.

In another aspect, the invention provides a method of identifying a candidate compound that ameliorates a bipolar disorder. The method involves contacting a cell that expresses a nucleic acid molecule encoding a proteasomal polypeptide with a candidate compound, and comparing the level of expression of the nucleic acid molecule in the cell contacted by the candidate compound with the level of expression in a control cell not contacted by the candidate compound, where an increase in expression of the nucleic acid molecule encoding a proteasomal polypeptide identifies the candidate compound as a candidate compound that ameliorates a bipolar disorder. In one preferred embodiment, the increase in expression is an increase in transcription or an increase in translation.

In another aspect, the invention features a pharmaceutical composition comprising a proteasomal polypeptide or portion thereof, selected from the group consisting of a Sec61 gamma, protein-L-isoaspartate (D-aspartate) O-methyltransferase, F-box only protein 9, and proteasome subunit z in a pharmaceutically acceptable carrier.

In another aspect, the invention features a pharmaceutical composition including a nucleic acid molecule encoding a proteasomal polypeptide, or portion thereof, selected from the group consisting of a Sec61 gamma, protein-L-isoaspartate (D-aspartate) O-methyltransferase, F-box only protein 9, and proteasome subunit z in a pharmaceutically acceptable carrier.

In a related aspect, the invention provides a method of identifying a candidate compound that ameliorates a bipolar disorder. The method involves contacting a cell that expresses a proteasomal polypeptide with a candidate compound, and comparing the level of expression of the polypeptide in the cell contacted by the candidate compound with the level of polypeptide expression in a control cell not contacted by the candidate compound, where an increase in the expression of a proteasomal polypeptide identifies the candidate compound as a candidate compound that ameliorates a bipolar disorder. In preferred embodiments, the increase in expression is assayed using an immunological assay, an enzymatic assay, or a radioimmunoassay.

In another aspect, the invention provides a method of identifying a candidate compound that ameliorates a bipolar disorder. The method involves contacting a cell that expresses a proteasomal polypeptide with a candidate compound, and comparing the biological activity of the polypeptide in the cell contacted by the candidate compound with the level of biological activity in a control cell not contacted by the candidate compound, where an increase in the biological activity of the proteasomal polypeptide identifies the candidate compound as a candidate compound that ameliorates a bipolar disorder. In another preferred embodiment, the increase in expression is assayed using an immunological assay, an enzymatic assay, or a radioimmunoassay.

In another aspect, the invention provides a method of identifying a candidate compound that ameliorates a bipolar disorder. The method involves a) contacting a cell with a candidate compound; b) obtaining a nucleic acid from the cell; c) contacting a microarray of a previous aspect with the nucleic acid; and d) detecting an increase in expression level of a nucleic acid molecule encoding a proteasomal polypeptide in the cell contacted with the candidate compound compared to a control cell, where the increase identifies the candidate compound as a candidate compound that ameliorates a bipolar disorder.

In another related aspect, the invention provides a method of identifying a candidate compound that ameliorates a bipolar disorder. The method involves the steps of a) contacting a microarray of a previous aspect with a candidate compound; and b) detecting binding of the candidate compound to a proteasomal polypeptide, where the binding identifies the compound as a candidate compound that ameliorates a bipolar disorder.

In another aspect, the invention features a collection of primer sets, each of the primer sets comprising at least two, four, six, eight, ten, or twenty primers that bind to a nucleic acid molecule encoding a proteasomal polypeptide selected from the group consisting of a Sec61 gamma, protein-L-isoaspartate (D-aspartate) O-methyltransferase, F-box only protein 9, and proteasome subunit z under high stringency conditions, the collection comprising at least two, four, six, eight, ten, or twenty primer sets.

In another aspect, the invention features a nucleic acid library comprising at least two, four, six, eight, ten, or twenty proteasomal nucleic acid molecules, wherein at least 90%, 95%, 100% of the nucleic acid molecules in the library are proteasomal nucleic acid molecules. In preferred embodiments, the nucleic acid molecules in the library are carried in a vector. In other preferred embodiments, each of the nucleic acid molecules is fused to a reporter gene.

In another aspect, the invention provides a method of identifying a candidate compound that ameliorates a bipolar disorder. The method involves a) contacting a cell comprising one member of the library of the previous aspect; and b) measuring the expression of the reporter gene; and c) comparing the level of reporter gene expression in the cell contacted with the candidate compound with a control cell not contacted with the candidate compound, where an increase in the level of the reporter gene expression identifies the candidate compound as a candidate compound that ameliorates a bipolar disorder.

In preferred embodiments of the previous aspects, a proteasomal polypeptide or nucleic acid molecule is any one or any combination of the following: a Sec61 gamma, protein-L-isoaspartate (D-aspartate) O-methyltransferase, F-box only protein 9, and proteasome subunit z polypeptide or nucleic acid molecule.

In another aspect, the invention features a microarray comprising at least two, ten, twenty, fifty, one hundred, two hundred, or three hundred nucleic acid molecules listed in Table 4, or fragments thereof, bound to a solid support, where at least 90%, 95%, or 100% of the nucleic acids bound to the support are nucleic acid molecules listed in Table 4.

In another aspect, the invention features a microarray comprising at least two, ten, twenty, fifty, one hundred, two hundred, or three hundred polypeptides listed in Table 4, or fragments thereof, bound to a solid support, where at least 90%, 95%, or 100% of the polypeptides on the support are polypeptides listed in Table 4.

In another aspect, the invention features a method of diagnosing a patient having, or having a propensity to develop, a bipolar disorder. The method involves determining the level of expression of a nucleic acid listed in Table 4 in a patient sample, where an alteration in the level of expression relative to the level of expression in a control sample, indicates that the patient has or has a propensity to develop a bipolar disorder. In one preferred embodiment, the patient sample is a blood sample. In another preferred embodiment, the level of expression is determined using a microarray or quantitative real-time PCR.

In another aspect, the invention provides a method of diagnosing a patient having, or having a propensity to develop, a bipolar disorder. The method involves determining the level of expression of a polypeptide encoded by a nucleic acid listed in Table 4 in a patient sample, where an altered level of expression relative to the level of expression in a control sample, indicates that the patient has or has a propensity to develop a bipolar disorder. In one preferred embodiment, the patient sample is a blood sample. In another preferred embodiment, the level of expression is determined using a microarray. In other embodiments, the level of expression is determined in an immunological or enzymatic assay.

In another aspect, the invention provides a method of monitoring a patient having a bipolar disorder. The method involves determining the level of expression of a nucleic acid or polypeptide listed in Table 4 in a patient sample, where an alteration in the level of expression relative to the level of expression in a control sample indicates the severity of a bipolar disorder in the patient. In one embodiment, the control sample is a normal patient sample or a reference sample taken from the patient. In one preferred embodiment, the patient sample is a blood sample. In another preferred embodiment, the patient is being treated for a bipolar disorder.

In another aspect, the invention provides a diagnostic kit for the diagnosis of a bipolar disorder comprising a nucleic acid molecule, or fragment thereof, selected from the group consisting of those listed in Table 4.

In another aspect, the invention provides a method of treating or preventing a bipolar disorder. The method involves administering to a patient in need of such treatment an effective amount of a pharmaceutical composition comprising a polypeptide encoded by a nucleic acid listed in Table 4.

In a related aspect, the invention provides a method of treating or preventing a bipolar disorder. The method involves administering to a patient in need of such treatment an effective amount of a pharmaceutical composition comprising a nucleic acid molecule selected from the group consisting of those listed in Table 4.

In another related aspect, the invention provides a method of treating or preventing a bipolar disorder. The method involves administering to a patient in need of such treatment an effective amount of a pharmaceutical composition that increases expression of a polypeptide encoded by a nucleic acid selected from the group consisting of those listed in Table 4.

In another related aspect, the invention provides a method of identifying a candidate compound that ameliorates a bipolar disorder. The method involves contacting a cell that expresses a nucleic acid molecule selected from the group consisting of those listed in Table 4 with a candidate compound, and comparing the level of expression of the nucleic acid molecule in the cell contacted by the candidate compound with the level of expression in a control cell not contacted by the candidate compound, where an alteration in expression of the nucleic acid molecule identifies the candidate compound as a candidate compound that ameliorates a bipolar disorder. In preferred embodiments, the alteration in expression is an alteration in transcription or translation.

In another related aspect, the invention features a method of identifying a candidate compound that ameliorates a bipolar disorder. The method involves contacting a cell expressing a polypeptide encoded by a nucleic acid selected from the group consisting of those listed in Table 4 with a candidate compound, and comparing the level of expression of the polypeptide in the cell contacted by the candidate compound with the level of polypeptide expression in a control cell not contacted by the candidate compound, where an alteration in the expression of a polypeptide encoded by a nucleic acid listed in Table 4 identifies the candidate compound as a candidate compound that ameliorates a bipolar disorder. In preferred embodiments, the alteration in expression is assayed using an immunological assay, an enzymatic assay, or a radioimmunoassay.

In a related aspect, the invention features a method of identifying a candidate compound that ameliorates a bipolar disorder. The method involves contacting a cell that expresses a polypeptide encoded by a nucleic acid selected from the group consisting of those listed in Table 4 with a candidate compound, and comparing the biological activity of the polypeptide in the cell contacted by the candidate compound with the level of biological activity in a control cell not contacted by the candidate compound, where an alteration in the biological activity of the polypeptide identifies the candidate compound as a candidate compound that ameliorates a bipolar disorder. In preferred embodiments, the alteration in expression is assayed using an immunological assay, an enzymatic assay, or a radioimmunoassay.

In another aspect, the invention features a method of identifying a candidate compound that ameliorates a bipolar disorder. The method involves a) contacting a cell with a candidate compound; b) obtaining a nucleic acid from the cell; c) contacting a microarray of a previous aspect with the nucleic acid; and d) detecting an alteration in expression level of a nucleic acid molecule encoding a polypeptide selected from the group consisting of those listed in Table 4 in the cell contacted with the candidate compound compared to a control cell, where the alteration identifies the candidate compound as a candidate compound that ameliorates a bipolar disorder.

In another aspect, the invention provides a method of identifying a candidate compound that ameliorates a bipolar disorder. The method involves a) contacting a microarray of a previous aspect with a candidate compound; and b) detecting binding of the candidate compound to a polypeptide selected from the group consisting of those listed in Table 4, where the binding identifies the compound as a candidate compound that ameliorates a bipolar disorder.

In another aspect, the invention provides a collection of primer sets, each of the primer sets comprising at least two, ten, twenty, fifty, one hundred, two hundred, or three hundred primers that bind to a nucleic acid molecule selected from the group consisting of those listed in Table 4.

In another aspect, the invention provides a purified nucleic acid library comprising at least two nucleic acid molecules selected from the group consisting of those listed in Table 4, where at least 90% of the nucleic acid molecule in the library are listed in Table 4. In one embodiment, the nucleic acid molecules in the library are carried in a vector. In another embodiment, each of the nucleic acid molecules is fused to a reporter gene.

In a related aspect, the invention features a method of identifying a candidate compound that ameliorates a bipolar disorder. The method involves: a) contacting a cell comprising one member of the library a previous aspect; b) measuring the expression of the reporter gene; and c) comparing the level of reporter gene expression in the cell contacted with the candidate compound with a control cell not contacted with the candidate compound, where an alteration in the level of the reporter gene expression identifies the candidate compound as a candidate compound that ameliorates a bipolar disorder.

In various embodiments of any of the previous aspects, the nucleic acid molecule is any one or more of the nucleic acid molecules listed in Table 2 or 4. Generally speaking, for any nucleic acid molecule listed in Table 4, which is identified as increased in a patient having a bipolar disorder, a candidate compound that decreases the expression or biological activity of the nucleic acid molecule or its encoded polypeptide is useful in the methods of the invention. Similarly, for any nucleic acid molecule listed in Table 2 or 4, which is identified as decreased in a patient having a bipolar disorder, a candidate compound that increases the expression or biological activity of the nucleic acid molecule or its encoded polypeptide is useful in the methods of the invention. In various embodiments of any of the previous aspects, the expression of a nucleic acid molecule is determined using standard methods including microarrays or quantitative real-time PCR.

By “a bipolar disorder” is meant a mood or affective disorder characterized by pathological mood swings from mania to depression. The diagnostic criteria for a bipolar disorder (e.g., bipolar I: mania and depression; bipolar II: hypomania and depression; bipolar III: cyclothymic disorders; bipolar IV: hypomania or mania precipitated by antidepressant drugs; bipolar V: depressed patient with bipolar relatives; and bipolar VI: mania without depression) are known to the skilled artisan, and are described in the Diagnostic and Statistical Manual of Mental Disorders, DSM-IV, 1994, American Psychiatric Association.

By “nuclear encoded mitochondrial energy metabolism nucleic acid molecule” is meant a polynucleotide, or fragment thereof, that naturally occurs in the nucleus and encodes a polypeptide that localizes to the mitochondria or that functions in mitochondrial energy metabolism.

By “nuclear encoded mitochondrial energy metabolism polypeptide” is meant a protein, or fragment thereof, that functions in mitochondrial energy metabolism and is encoded by a nucleic acid molecule that naturally occurs in the cell nucleus. In some embodiments, the polypeptide functions in oxidative phosphorylation. Specifically excluded by this definition are mitochondrial genome encoded polypeptides.

By “proteasomal nucleic acid molecule” is meant a nucleic acid molecule that encodes a polypeptide that localizes to the proteasome or that functions in proteasomal degradation.

By “proteasomal polypeptide” is meant a polypeptide that localizes to the proteasome or that functions in proteasomal degradation. In preferred embodiments, the polypeptide functions in the ATP-dependent 19S proteasome or 20S proteasome.

By “microarray” is meant an organized collection of at least two nucleic acid molecules or polypeptides affixed to a solid support. In some embodiments, a nucleic acid microarray is composed of oligonucleotides having at least a portion (e.g., 10, 15, 18, 20, 21, 22, 23, 24, 25, 26, 27, 28, 29, 30, 40, 50, 60, 70, 80, 90, or 100 nucleotides) of two or more nucleic acid sequences listed in Table 2 or Table 4. A polypeptide microarray contains at least a polypeptide (e.g., 10, 20, 30, 40, 50, 75, or 100 amino acids) encoded by a nucleic acid listed in Table 2 or Table 4. A microarray contains at least 2, 5, 10, 25, 50, 75, 100, 150, 200, 250, or 300 polypeptide or nucleic acid molecule members.

By “portion” is meant a fragment of a protein or nucleic acid that is substantially identical to a reference protein or nucleic acid. In some embodiments the portion retains at least 50% 75%, or 80%, or more preferably 90%, 95%, or even 99% of the biological activity of the reference protein or nucleic acid described herein.

By “isolated nucleic acid molecule” is meant a nucleic acid (e.g., a DNA) that is free of the genes that, in the naturally occurring genome of the organism from which the nucleic acid molecule of the invention is derived, flank the gene. The term therefore includes, for example, a recombinant DNA that is incorporated into a vector; into an autonomously replicating plasmid or virus; or into the genomic DNA of a prokaryote or eukaryote; or that exists as a separate molecule (for example, a cDNA or a genomic or cDNA fragment produced by PCR or restriction endonuclease digestion) independent of other sequences. In addition, the term includes an RNA molecule that is transcribed from a DNA molecule, as well as a recombinant DNA that is part of a hybrid gene encoding additional polypeptide sequence.

By “polypeptide” is meant any chain of amino acids, regardless of length or post-translational modification (for example, glycosylation or phosphorylation).

By an “isolated polypeptide” is meant a polypeptide of the invention that has been separated from components that naturally accompany it. Typically, the polypeptide is isolated when it is at least 60%, by weight, free from the proteins and naturally occurring organic molecules with which it is naturally associated. Preferably, the preparation is at least 75%, more preferably at least 90%, and most preferably at least 99%, by weight, a polypeptide of the invention. An isolated polypeptide of the invention may be obtained, for example, by extraction from a natural source, by expression of a recombinant nucleic acid encoding such a polypeptide; or by chemically synthesizing the protein. Purity can be measured by any appropriate method, for example, column chromatography, polyacrylamide gel electrophoresis, or by HPLC analysis.

By “transformed cell” is meant a cell into which (or into an ancestor of which) has been introduced, by means of recombinant DNA techniques, a polynucleotide molecule.

By “positioned for expression” is meant that the polynucleotide of the invention (e.g., a DNA molecule) is positioned adjacent to a DNA sequence that directs transcription and translation of the sequence (i.e., facilitates the production of, for example, a recombinant polypeptide, or an RNA molecule).

By “purified antibody” is meant an antibody that is at least 60%, by weight, free from proteins and naturally-occurring organic molecules with which it is naturally associated. Preferably, the preparation is at least 75%, more preferably 90%, and most preferably at least 99%, by weight, antibody. A purified antibody of the invention may be obtained, for example, by affinity chromatography using a recombinantly-produced polypeptide of the invention and standard techniques.

By “specifically binds” is meant a compound or antibody which recognizes and binds a polypeptide of the invention, but that does not substantially recognize and bind other molecules in a sample, for example, a biological sample, which naturally includes a polypeptide of the invention.

By “derived from” is meant isolated from or having the sequence of a naturally-occurring sequence (e.g., a cDNA, genomic DNA, synthetic, or combination thereof).

By “immunological assay” is meant an assay that relies on an immunological reaction, for example, antibody binding to an antigen. Examples of immunological assays include ELISAs, Western blots, immunoprecipitations, and other assays known to the skilled artisan.

By “differentially expressed” is meant an alteration in the expression level of a nucleic acid or polypeptide. This difference may be either an increase or a decrease in expression, when compared to control conditions. Preferably, the increase or decrease is at least 10%, 20%, 30%, 40%, 50%, 60%, 70%, 80%, 90%, or even 100%.

By “therapeutic compound” is meant a substance that affects the function of an organism. Such a compound may be, for example, an isolated naturally occurring, semi-synthetic, or synthetic agent. For example, a therapeutic compound may be a drug that ameliorates the symptoms of a bipolar disorder in a patient as assayed by standard psychiatric diagnostic criteria (e.g., Diagnostic and Statistical Manual of Mental Disorders, DSM-IV, 1994, American Psychiatric Association). A therapeutic compound may decrease, suppress; attenuate, diminish, arrest, or stabilize the development or progression of a mental disease or disorder in a human.

By “effective amount” is meant one or more dosages of a therapeutic agent sufficient to ameliorate, stabilize, or prevent the symptoms of a disease or disorder as evaluated using standard diagnostic methods. An effective amount of a therapeutic compound ameliorates or reduces the symptoms of a bipolar disorder as assayed by psychiatric diagnostic criteria (Diagnostic and Statistical Manual of Mental Disorders, DSM-IV, 1994, American Psychiatric Association); by increasing the expression of a nucleic acid or polypeptide sequence listed in Table 2 or altering the expression of a nucleic acid or polypeptide sequence listed in Table 4 by at least 10%, 20%, 30%, 40%, 50%, 60%, or even by as much as 70%, 80%, 90%, 95%, or 100% as compared to the expression in an untreated control patient having a bipolar disorder; by increasing the activity of a mitochondrial energy metabolism or proteasomal pathway by at least 10%, 20%, 30%, 40%, 50%, 60%, or even by as much as 70%, 80%, 90%, or 100% as compared to the activity of the pathway in an untreated control patient having a bipolar disorder; or by assaying brain function using standard spectroscopic methods (e.g., U.S. Pat. Nos.:6,400,978 or 6,104,943 hereby incorporated by reference). The activity of a mitochondrial energy metabolism pathway is assayed using standard methods (e.g., Adenosine 5-triphosphate (ATP) bioluminescent somatic cell assay kit, Sigma-Aldrich (St. Louis, Mo.) Catalog No.:FL-ASC; or Kowaltowski et al., J Biol Chem 277:42802-7, 2002). The activity of a proteasomal pathway is assayed using standard methods (e.g., 20S Proteasome Assay Kit (Affiniti Research Products, Exeter, United Kingdom, Catalog No.: PW8920).

By “a collection” is meant a group having more than one member. The group may be composed of 2, 4, 5, 8, 10, 20, 30, 40, 50, 60, 70, 80, 90, 100, 125, 150, 200, 250, or 300 polypeptide, nucleic acid molecule, or chemical compound members.

By “a library” is meant a collection of polypeptide, nucleic acid, or chemical compound members. A library may contain at least 2, 10, 25, 50, 100, 150, 200, 250, 300, 400, 500, 750, or more members.

The invention features methods and compositions for the diagnosis, monitoring, and treatment of bipolar disorder in human patients, as well as methods and compositions for the identification of new candidate therapeutics.

Other features and advantages of the invention will be apparent from the detailed description, and from the claims.

### BRIEF DESCRIPTION OF THE DRAWINGS

FIG. 1 is a diagram showing that nuclear encoded mitochondrial energy metabolism gene expression is decreased in patients having bipolar disorder. Specifically, decreased expression is identified in genes encoding polypeptides that function in oxidative phosphorylation in mitochondrial complex I, II, III, IV, and V.

FIG. 2 is a diagram showing that proteasomal gene expression is decreased in patients having bipolar disorder. Specifically, decreased expression is identified in genes encoding polypeptides that function in the ATP-dependent 19S proteasome and in the catalytic core of the 20S proteasome.

FIGS. 3A and 3B are diagrams showing sample clustering according to gene expression pregulation. All genes with a standard deviation above 4% of the mean of their expression value, and having ‘present’ calls in at least 20% of samples were used for clustering (n=216 genes). Significant clustering of bipolar disorder samples was observed (p=0.005).

FIG. 3B is a diagram showing sample clustering with genes known to be involved in complexes I-V of the mitochondrial respiratory chain and ‘present’ in at least 20% of samples were used for clustering (n=72 genes). Significant clustering of bipolar disorder samples (p=0.004) and of control samples (p=0.024) was observed. Redundant probe sets were excluded from clustering analysis. Valproic acid and lithium carbonate treatment is denoted in the figure. Black boxes denote bipolar disorder. Hatched boxes denote schizophrenia. White boxes denote control. L and V denote treatment with lithium and valproic acid, respectively.

FIGS. 4A-4H are graphs showing that gene expression is decreased in four exemplary genes as detected by quantitative real time PCR. Four genes were selected for verification in hippocampal tissue (A-D) and for exploratory purposes in tissue from Brodmann area 9 in the frontal cortex (E-H): A, the oligomycin sensitivity conferring protein (OSCP), a subunit of the mitochondrial ATP synthase, B, the mitochondrial cytochrome c oxidase subunit COX VIIb, C, the proteasome alpha 3 subunit and D, the proteasome beta 4 subunit. The expression of each gene was normalized over filamin A. Co, control, BD, bipolar disorder. Average ±SEM is shown, *p<=0.05.

### DESCRIPTION OF THE INVENTION

The invention features methods and compositions for the treatment, diagnosis, and monitoring of bipolar disorder in human patients. The disease mechanism of bipolar disorder, a serious psychiatric disorder with recurrent episodes of depression and mania, is unknown. In a gene array study that examined the regulation of 13,000 genes in patients with bipolar disorder, expression of nuclear mRNA encoding mitochondrial proteins was found to be significantly decreased in the hippocampus in bipolar disorder. These changes were specific to bipolar disorder, and were not observed in patients with schizophrenia. A pronounced and extensive decrease in the expression of genes regulating oxidative phosphorylation and the ATP-dependent process of proteasome degradation was identified in bipolar disorder. These results indicate that the primary defect in bipolar disorder is in nuclear genes that encode mitochondrial proteins. In contrast, spectroscopic studies had linked bipolar disorder to defects in the mitochondrial genome. (Kato et al., Journal of Affective Disorders 27:53-9,1993; Kato et al., Bipolar Disorders 2:180-90, 2000; Kato et al., Journal of Neuropsychiatry & Clinical Neurosciences 10:133-147, 1998).

These experiments were carried out as follows.

Gene Chip Preparation

Hippocampal brain tissue was obtained from the Harvard Brain Tissue Resource Center (http://www.brainbank.mclean.org:8080/) from patients with bipolar disorder (n=9), schizophrenic patients (n=8), and control patients free of mental illness (Table 1). The diagnosis of each patient was verified by two psychiatrists who reviewed the donors' medical records.

Human hippocampal RNA was prepared using standard methods provided by AFFYMETRIX (Santa Clara, Calif.). RNA was extracted from 50-100 mg tissue with RNAGENT KIT (Promega, Madison Wis.), an RNA isolation kit. RNA quality was assessed with a BIOANALYZER (Agilent Technologies, Palo Alto, Calif.). Eight μg total RNA was used for cDNA synthesis with the SUPERSCRIPT double-stranded cDNA synthesis kit (Invitrogen Corp., Carlsbad, Calif.), and in vitro transcription was performed with the ENZO-IVT kit (Enzd Biochem, Farmingdale, N.Y.). Biotin-labeled RNA from each hippocampus was fragmented and hybridized to an HG-U95Av2 array (Affymetrix) during an overnight incubation at 45° C. in hybridization buffer containing 50 pM Control Oligo B2 (Affymetrix), 1× eukaryotic hybridization controls (AFFYMETRIX 1.5 pM bioB, 5 pM bioC, and 25 pM bioD probes that hybridize to bioB, bioC, and bioD biotin synthesis genes of E. coli; and 100 pM cre probe that hybridizes to the cre of P1 bacteriophage), 0.1 μg/μl Herring sperm DNA, 0.5 μg/μl acetylated Bovine Serum Albumin, 100 mM 2-Morpholinoethanesulfonic acid, 1 M sodium, 20 mM EDTA, 0.01% Tween 20 and 50 ng/μl fragmented sample (biotinylated cRNA). The hybridized RNA was stained with streptavidin-phycoerythrin (Molecular Probes, Eugene, Oreg.), incubated with a biotinylated anti-streptavidin antibody (Vector Laboratories (Burlingame, Calif.) and then stained a second time with streptavidin-phycoerythrin. Twenty-eight chips were thus obtained, containing hybridized and stained RNA from each hippocampus (10 controls, 9 bipolar disorders and 9 schizophrenics). Gene expression levels were detected using a GeneArray® Scanner (Affymetrix, Santa Clara, Calif.)

Data Analysis

The dChip program, which was developed by Wong et al. of the Harvard School of Public Health is publicly available at the dChip website, was used to analyze the data. Model-based expression was performed only on “perfect match” data. The term “perfect match” refers to sample RNA hybridization to a 25-base oligonucleotide bound to the chip. The sequence of these 25-base oligonucleotides is identical to the nucleic acid sequence of a human gene present in a human genome database. Control cells present on the chip contained oligonucleotides having altered sequences. These “mismatch” oligonucleotides contained one nucleotide (nucleotide 13) that was mutated relative to the correct sequence present in a human genome database. The type of analysis chosen did not take mismatch oligonucleotides into consideration (Li, et al., Proc. Natl. Acad. Sciences USA. 98, 31-36, 2001).

Data obtained with the dChip program were prepared for GenMAPP program analysis (Dahlquist et al.,Nature Genetics 31:19-20, 2002) using the following criteria: p<0.02, fold induction above 1.1, and “present calls” in more than 50% of the samples. The term “present calls” refers to an analytical method that determines whether or not a particular gene is considered to be present (i.e., expressed) in a particular sample based on the specificity of sample binding. A gene is considered to be present when a sample binds specifically to a gene-derived oligonucleotide. A gene is considered to be absent when the sample fails to bind to a gene-derived oligonucleotide sequence, or when there is no specific binding to the gene-derived oligonucleotide (i.e., no significant difference in binding of the sample to a perfect match oligonucleotide versus binding to a mismatch oligonucleotide). A control sample with average intensity was chosen for normalization.

The MAPPfinder program (http://www.genmapp.org) identifies regulatory trends among groups of genes that are organized by biological process, molecular function, or cellar component, as defined by the Gene Ontology (GO) consortium (http://www.geneontology.org). For algorithm verification, data were also computed with the Affymetrix Data Mining Tool (version 3.0).

Sample information and quality control criteria are presented in Table 1.

TABLE 1
Demographics and Microarray Quality Control Data

| Case | | | | | | 3′5′ | 3′5′ | | Cause of | |
|---|---|---|---|---|---|---|---|---|---|---|
| No. | Diag | H | Sex | Age | Pmi | ga | βa | % P | death | Psychoactive medication use |
| B | C | R | F | 70 | 15 | 2.2 | 3.2 | 45.1 | Cardiac | — |
| 3806 | | | | | | | | | Arrest | |
| B | C | R | F | 78 | 14.1 | 3.1 | 3.9 | 45 | MI | — |
| 3898 | | | | | | | | | | |
| B | C | L | M | 29 | 18.2 | 2.0 | 2.5 | 43.4 | Renal | — |
| 4605 | | | | | | | | | Failure | |
| B | C | L | F | 74 | 12.5 | 3.4 | 3.6 | 37.3 | Renal | — |
| 4737 | | | | | | | | | Failure | |
| B | C | L | M | 54 | 24.2 | 2.3 | 2.7 | 47.1 | Cardiac | — |
| 4751 | | | | | | | | | Arrest | |
| B 4810 | C | L | F | 62 | 16.4 | 1.6 | 2.1 | 45.9 | Lung CA | — |
| B 4853 | C | L | F | 70 | 22.5 | 2.0 | 2.6 | 43.3 | Liver CA | — |
| B 4932 | C | R | M | 67 | 22.3 | 1.5 | 2 | 47.4 | Cardiac Arrest | — |
| B 5074 | C | R | M | 79 | 20.9 | 1.8 | 2.6 | 48.3 | Pancreas CA | — |
| B 5082 | C | R | F | 78 | 23.9 | 1.9 | 2.1 | 45.4 | Breast CA | — |
| B | BD | R | F | 64 | 11.0 | 2.1 | 2.5 | 46.3 | Respiratory | Trifluoperazine 40 mg qd |
| 3817 | | | | | | | | | Failure | |
| B | BD | L | F | 80 | 11.6 | 2.0 | 2.6 | 44.9 | Cerebrovascular | Perphenazine 4 m qam, |
| 4069 | | | | | | | | | Accident | Benztropine, Valproate 1000 mg qd |
| B | BD | L | F | 76 | 22.8 | 2.5 | 3.0 | 41.3 | Cardiopulmonary | LiCO3 300 mg bid |
| 4403 | | | | | | | | | Failure | |
| B 4462 | BD | R | M | 50 | 30.5 | 3.4 | 3.8 | 42 | Cardiac Arrest | ? |
| B 4464 | BD | L | M | 74 | 24.8 | 2.8 | 3.3 | 39.1 | Pneumonia | Depakote 1000 mg bid; Quetiapine 425 mg qd |
| B | BD | L | M | 25 | 12.6 | 2.6 | 2.7 | 39 | Pulmonary | Sertraline, Trazodone, Gabapentin, LiCO3 |
| 4661 | | | | | | | | | Edema | |
| B 4914 | BD | R | F | 73 | 20.8 | 2.2 | 2.5 | 42.9 | Sepsis | Risperidone 0.5 mg qd, |
| | | | | | | | | | | Carbamazepine 100 mg bid |
| B 4961 | BD | R | M | 74 | 14.3 | 2.6 | 3.3 | 42.2 | Pneumonia | Lithium, Divalproex sodium, Olanzapine |
| B 5044 | BD | R | F | 73 | 17 | 2.7 | 3.5 | 38.6 | Renal Failure | LiCO3, Divalproex sodium, Risperidone 2 mg qd |
| B | SA | L | F | 78 | 13.4 | 2.7 | 3.6 | 44.1 | Sinus Node | Lithium, Haloperidol |
| 4190 | | | | | | | | | Disease | |
| B | S | R | M | 26 | 16 | 1.6 | 1.6 | 47.4 | Suicide by | Prolixin Dec 25 mg im q2w |
| 4238 | | | | | | | | | Hanging | |
| B | S | L | M | 80 | 11 | 2.5 | 3.4 | 47.1 | Cardiopulmonary | Thioridazine 10 mg qd |
| 4469 | | | | | | | | | Failure | |
| B 4875 | SA | R | F | 55 | 18 | 1.7 | 1.7 | 47.7 | Cancer | Depakote 250 bid, Prolixin IM q 1m |
| B 4907 | SA | R | F | 73 | 24 | 3.0 | 2.8 | 38.0 | Lung CA | Prolixin 15 mg qd |
| B 5047 | S | R | M | 63 | 22.3 | 2.4 | 3.8 | 40.0 | Cardiac Arrest | Clozapine 400 mg qd, Clonazepam 0.5 mg bid |
| B | S | R | F | 72 | 21.7 | 1.5 | 2.0 | 47.4 | Cancer | Risperidone 4 mg qd, Benztropine 0.5 mg prn, |
| 5100 | | | | | | | | | | Paroxetine 50 mg qd |
| B | S | L | M | 49 | 24.5 | 1.5 | 1.6 | 47.1 | Acute | Haldol Decanoate 200 mg q 4w |
| 5115 | C | 5/5 | 6/4 | 66.1 | 19.00 | 2.18 | 2.73 | 44.82 | Respiratory Failure | |
| | BD | 4/5 | 5/4 | 65.4 | 18.38 | 2.54 | 3.03 | 41.81* | | |
| | S | 3/5 | 4/4 | 62.0 | 18.86 | 2.11 | 2.57 | 44.85 | | |

Table Legend
Diag = Diagnosis;
C = control,
S = schizophrenic;
BD = bipolar disorder
H = Brain Hemisphere;
L = left,
R = right
Sex; F = female, M = male
CPZ = chloropromazine equivalency values
PMI = post mortem interval expressed in hours
3′5′ G = 3′/5′ ratios for GAPDH
3′5′ β = 3′/5′ ratios for β-actin
% P = present calls of all genes analyzed
SF = scaling factor-

Samples were matched for age, post mortem interval, gender and brain hemisphere. No significant differences were observed in the quality control criteria provided by the Data Mining Tool (Affymetrix) and dChip analyses (3′/5′ ratios for GAPDH and β-actin, scaling factor-SF, background), or in the 28S/18S ratio obtained with the BIOANALYZER. A significant difference was found in the gene present calls, which were lower (p<=0.05) in the bipolar disorder samples, indicating that these genes were down regulated in patients having a bipolar disorder.

Gene Expression Changes In Bipolar Disorder

Gene expression was considered to be significantly different from the control group when mean values were at least 1.2 fold different (p=0.01). Employing these statistical thresholds, the expression of forty-three genes was decreased in bipolar disorder (Table 2).

TABLE 2
Decreased Gene Expression in Bipolar Disorder (p = 0.01)

| | Gene | Map Location | fold | P value | Pres % |
|---|---|---|---|---|---|
| | Mitochondrial | | | | |
| 1 | ATP synthase, mitochondrial F0 complex, subunit c, isoform 3 | 2q31.1 | −1.63 | 0.0006 | 100 |
| 2 | VDAC1 pseudogene, porin protein, isoform 1 | X | −1.41 | 0.0007 | 94 |
| 3 | Ubiquinone-binding protein | 5q31.1 | −1.37 | 0.0011 | 100 |
| 4 | ATP synthase, mitochondrial F0 complex, subunit d | 17q25 | −1.67 | 0.0011 | 100 |
| 5 | Mitochondrial ribosomal protein L3 | 3q21-q23 | −1.46 | 0.0011 | 100 |
| 6 | Cytochrome c oxidase subunit VIIb | Xq13.2 | −1.58 | 0.0013 | 100 |
| 7 | ATP synthase, mitochondrial F0 complex, subunit f, isoform 2 | 7q11.21 | −1.48 | 0.0016 | 100 |
| 8 | Dynamin 1-like | 12p12.1 | −1.66 | 0.0016 | 68 |
| 9 | Voltage-dependent anion channel 2; porin | 10q22 | −1.40 | 0.0018 | 100 |
| 10 | Cytochrome c oxidase subunit VIIa polypeptide 2 (liver) | 6q12 | −1.42 | 0.0021 | 100 |
| 11 | ATP synthase, mitochondrial F1 complex, O subunit (OSCP) | 21q22.11 | −1.53 | 0.0025 | 100 |
| 12 | Voltage-dependent anion channel 1; porin | 5q31 | −1.49 | 0.0029 | 100 |
| 13 | Single-stranded DNA binding protein | 7q34 | −1.44 | 0.0030 | 94 |
| 14 | Fumarate hydratase | 1q42.1 | −1.47 | 0.0036 | 100 |
| 15 | Solute carrier family 25, member 4 | 4q35 | −1.53 | 0.0038 | 100 |
| 16 | ATP synthase, mitochondrial F1 complex, gamma polypeptide 1 | 10q22-q23 | −1.46 | 0.0045 | 100 |
| 17 | NADH dehydrogenase (ubiquinone) 1, alpha/beta subcomplex, 1, 8 kDa | 16p11.2 | −1.45 | 0.0053 | 100 |
| 18 | 3-oxoacid CoA transferase | 5p13 | −1.62 | 0.0089 | 100 |
| | Energy metabolism | | | | |
| 19 | UDP-glucose pyrophosphorylase 2 | 2p14-p13 | −1.44 | 0.0019 | 100 |
| 20 | ATPase, lysosomal 70 kDa, V1 subunit A, isoform 1 | 3q13.31 | −1.54 | 0.0043 | 89 |
| 21 | ATPase, lysosomal 34 kDa, V1 subunit D | 14 | −1.47 | 0.0056 | 100 |
| | Protein degradation | | | | |
| 22 | Sec61 gamma | 7p14.1 | −1.39 | 0.0009 | 100 |
| 23 | Proteasome (prosome, macropain) 26S subunit, ATPase, 6 | 14q22.1 | −1.49 | 0.0021 | 100 |
| 24 | Protein-L-isoaspartate (D-aspartate) O-methyltransferase | 6q24-q25 | −1.75 | 0.0065 | 100 |
| 25 | F-box only protein 9 | 6p12.3-p11.2 | −1.68 | 0.0077 | 100 |
| | Neurotransmission | | | | |
| 26 | Somatostatin | 3q28 | −2.78 | 0.0062 | 84 |
| 27 | Glutamic acid decarboxylase 67 | 2q31 | −1.80 | 0.0090 | 100 |
| | Structural proteins | | | | |
| 28 | Actin related protein 2/3 complex, subunit 3, 21 kDa | 12q24 | −1.49 | 0.0004 | 100 |
| 29 | Beta-tubulin, beta2 | | −1.47 | 0.0019 | 100 |
| 30 | Actin-related protein 2 homolog (yeast) | 2p14 | −1.50 | 0.0022 | 100 |
| | Others | | | | |
| 31 | Macrophage migration inhibitory factor (MIF) | | −1.35 | 0.0007 | 100 |
| 32 | Rho guanine nucleotide exchange factor (GEF) 4 | 2q22 | −1.39 | 0.0012 | 100 |
| 33 | FSHD region gene 1 | 4q35 | −1.42 | 0.0014 | 100 |
| 34 | Eukaryotic translation initiation factor 3 subunit 11 | 19q13.2 | −1.53 | 0.0021 | 100 |
| 35 | Ataxin-10 (spinocerebellar ataxia type 10 protein) | 22q13.31 | −1.67 | 0.0029 | 100 |
| 36 | UDP-GlcNAc:betaGal beta-1,3-N-acetylglucosaminyltransferase 6 | 11q12.1 | −1.50 | 0.0037 | 100 |
| 37 | Contactin 1; glycoprotein gp135 | 12q11-q12 | −1.77 | 0.0046 | 63 |
| 38 | Endosulfine alpha, a regulator of beta-cell K(ATP) channels | 1q21.1 | −1.50 | 0.0048 | 100 |
| 39 | Tyrosine 3-monooxygenase/tryptophan 5-monooxygenase activation protein | 8q23.1 | −1.54 | 0.0067 | 100 |
| 40 | Chromosome 1 open reading frame 15; KIAA0479 protein | 1q25 | −1.67 | 0.0074 | 94 |
| 41 | Arg protein tyrosine kinase binding protein | | −1.51 | 0.0076 | 73 |
| 42 | Fk506-Binding Protein, Alt. Splice 2 | | −1.47 | 0.0078 | 84 |
| 43 | Glutamic-oxaloacetic transaminase 1, soluble (aspartate aminotransferase 1) | 10q24.1-q25.1 | −1.61 | 0.0084 | 100 |

Interestingly, 18 of these genes (42%) encoded mitochondrial proteins, including subunits of the membrane-bound respiratory enzyme complexes that carry out oxidative phosphorylation in the mitochondrial inner membrane. The changes in gene expression observed in hippocampi from patients with bipolar disorder included a decrease in expression of one gene encoding a component of mitochondrial respiratory complex I, NADH dehydrogenase; a decrease in one gene encoding a component of complex IV, cytochrome c oxidase; and a decrease in five genes encoding components of complex V, ATP synthases.

Gene array analysis confirmed the results of studies (Heckers et al., Archives of General Psychiatry 59:521-529, 2002) showing that expression of the 67 kD isoform of glutamic acid decarboxylase (GAD), the biosynthetic enzyme for the inhibitory neurotransmitter GABA, is decreased in bipolar disorder.

In addition, a decrease in mRNA coding for the neuropeptide somatostatin was observed in hippocampi from patients with bipolar disorder. Somatostatin is expressed in the O-LM subtype of hippocampal interneurons (Freund et al., Hippocampus 6:347-470, 1996). Interestingly, this decrease was the most significant of all the forty-three differentially affected genes. This supports the hypothesis that a subset of hippocampal interneurons, located in the stratum oriens and terminating on apical dendrites of principal cells in conjunction with perforant pathway afferents, is abnormal in bipolar disorder.

Table 3 provides the function of the gene products encoded by these down regulated genes, their subcellular localization, and Genpept sequence identifiers. Genpept is a publicly available protein database associated with Genbank.

TABLE 3
Function of Down Regulated Genes (p < 0.01)

| Title | Accession No. | Localization | Function |
|---|---|---|---|
| tyrosine 3- | M86400 | cyoplasmic | activates tyrosine and tryptophan hydroxylases in the |
| monooxygenase/tryptophan | | | presence of ca(2+)/calmodulin-dependent protein |
| 5-monooxygenase activation | | | kinase ii, and strongly activates protein kinase c. is |
| protein, zeta polypeptide; | | | probably a multifunctional regulator of the cell |
| Human phospholipase A2 | | | signaling processes mediated by both kinases. activates |
| | | | the adp-ribosyltransferase (exos) activity of bacterial |
| | | | origin |
| eukaryotic translation | AB019392 | | binds to the 40s ribosome and promotes the binding of |
| initiation factor 3 subunit 11 | | | methionyl-trnai and mrna (by similarity) |
| VDAC1 pseudogene | AJ002428 | mitochondrial | |
| (voltage-dependent anion | | outer | |
| channel (VDAC) of the outer | | membrane | |
| mitochondrial membrane); | | | |
| porin protein, isoform 1 | | | |
| contactin 1; glycoprotein | Z21488 | peripheral | mediates cell surface interactions during nervous |
| gp135 | | plasma | system development. in association with cntnap1 |
| | | membrane; | seems to play a role in the formation of paranodal axo- |
| | | attached to the | glial junctions in myelinated peripheral nerves and may |
| | | membrane by | have a role in the signaling between axons and |
| | | a gpi-anchor | myelinating glial cells |
| chromosome 1 open reading | AB007948 | cytoplasmic | This gene product belongs to the nicotinamide |
| frame 15; KIAA0479 protein; | | | mononucleotide adenylyltransferase (NMNAT) |
| nicotinamide mononucleotide | | | enzyme family, members of which catalyze an |
| adenylyltransferase 2 | | | essential step in NAD (NADP) biosynthetic pathway. |
| fumarate hydratase | U59309 | mitochondrial | tricarboxylic acid cycle |
| solute carrier family 25 | J02966 | mitochondrial | catalyzes the exchange of adp and atp across the |
| (mitochondrial carrier; | | inner | mitochondrial inner membrane |
| adenine nucleotide | | membrane | |
| translocator), member 4 | | | |
| UDP-GlcNAc:betaGal beta- | AF029893 | type ii | can initiate the synthesis or the elongation of the linear |
| 1,3-N- | | membrane | poly-n-acetyllactosaminoglycans |
| acetylglucosaminyltransferase | | protein. golgi. | |
| 6; i-beta-1,3-N- | | | |
| acetylglucosaminyltransferase | | | |
| Homo sapiens beta 2; beta- | X02344 | | |
| tubulin | | | |
| ATPase, H+ transporting, | AA877795 | lysosomal | vacuolar ATPase |
| lysosomal 34 kDa, V1 subunit D | | | (V-ATPase), a multisubunit enzyme that mediates |
| | | | acidification of |
| | | | eukaryotic intracellular organelles. |
| low molecular mass | A1540957 | mitochondrial | component of the ubiquinol-cytochrome c reductase |
| ubiquinone-binding protein | | inner | complex (complex iii or cytochrome b-cl complex), |
| (9.5 kD); ubiquinol- | | membrane | |
| cytochrome c reductase | | | |
| complex ubiquinone-binding | | | |
| protein | | | |
| ATP synthase, H+ | U09813 | mitochondrial | ATP synthase, H+ transporting |
| transporting, mitochondrial | | inner | |
| F0 complex, subunit c | | membrane | |
| (subunit 9) isoform 3 | | | |
| ATPase, H+ transporting, | L09235 | vacuolar | catalytic subunit of the peripheral v1 complex of |
| lysosomal 70 kDa, V1 subunit | | | vacuolar atpase. v-atpase vacuolar atpase is responsible |
| A, isoform 1 | | | for acidifying a variety of intracellular compartments |
| | | | in eukaryotic cells |
| NADH dehydrogenase | AC002400 | mitochondrial | complex i is composed of about 30 different subunits |
| (ubiquinone) 1, alpha/beta | | inner | |
| subcomplex, 1, 8 kDa | | membrane | |
| glutamic-oxaloacetic | M37400 | cyoplasmic | 1-aspartate + 2-oxoglutarate = oxaloacetate + 1- |
| transaminase 1, soluble | | | glutamate |
| (aspartate aminotransferase 1) | | | |
| ARP2 actin-related protein 2 | AF006082 | cytoskeleton | part of a complex implicated in the control of actin |
| homolog (yeast); one of seven | | | polymerization in cells |
| subunits of the Arp2/3 protein | | | |
| complex; actin-related | | | |
| protein. | | | |
| ATP synthase, H+ | AF087135 | mitochondrial | this is one of the chains of the nonenzymatic |
| transporting, mitochondrial | | inner | component (cf(0) subunit) of the mitochondrial atpase |
| F0 complex, subunit d | | membrane | complex. |
| actin related protein 2/3 | AI525393 | cytoplasmic | part of a complex implicated in the control of actin |
| complex, subunit 3, 21 kDa | | | polymerization in cells |
| Identification of ArgBP1, an | X95677 | cytoskeleton | Arg protein tyrosine kinase binding protein |
| Arg protein tyrosine kinase | | | |
| binding protein that is the | | | |
| human homologue of a CNS- | | | |
| specific Xenopus gene | | | |
| Rho guanine nucleotide | AB029035 | | |
| exchange factor (GEF) 4 | | | |
| cytochrome c oxidase subunit | N50520 | mitochondrial | |
| VIIb | | inner | |
| | | membrane | |
| ATP synthase, H+ | X83218 | mitochondrial | |
| transporting, mitochondrial | | inner | |
| F1 complex, O subunit | | membrane | |
| (oligomycin sensitivity | | | |
| conferring protein) | | | |
| glutamate decarboxylase 1 | M81883 | | |
| (brain, 67 kDa) | | | |
| UDP-glucose | U27460 | cyoplasmic | plays a central role as a glucosyl donor in cellular |
| pyrophosphorylase 2 | | | metabolic pathways |
| voltage-dependent anion | L08666 | mitochondrial | forms a channel through the mitochondrial outer |
| channel 2; porin, | | outer | membrane that allows diffusion of small hydrophilic |
| mitochondrial | | membrane | molecules, the channel adopts an open conformation at |
| | | | low or zero membrane potential and a closed |
| | | | conformation at potentials above 30-40 mv. the open |
| | | | state has a weak anion selectivity whereas the closed |
| | | | state is cation-selective |
| mitochondrial ribosomal | X06323 | mitochondrial | belongs to the 13p family of ribosomal proteins |
| protein L3 | | | |
| protein-L-isoaspartate (D- | D25547 | cyoplasmic | catalyzes the methyl esterification of 1-isoaspartyl and |
| aspartate) O- | | | d-aspartyl residues in peptides and proteins that result |
| methyltransferase | | | from spontaneous decomposition of normal 1-aspartyl |
| | | | and 1-asparaginyl residues. it plays a role in the repair |
| | | | and/or degradation of damaged proteins |
| somatostatin | J00306 | secreted | somatostatin inhibits the release of somatotropin |
| single-stranded DNA binding | AA768912 | mitochondrial | this protein binds preferentially and cooperatively to |
| protein | | | ss-dna. probably involved in mitochondrial dna |
| | | | replication |
| FSHD region gene 1 | L76159 | | deleted in facioscapulohumeral muscular dystrophy |
| F-box only protein 9 | AL031178 | | probably recognizes and binds to some phosphorylated |
| | | | proteins and promotes their ubiquitination and |
| | | | degradation; The F-box proteins constitute one of the |
| | | | four subunits of |
| | | | the ubiquitin protein ligase complex called SCFs |
| endosulfine alpha, a regulator | X99906 | | endogenous ligand for sulfonylurea receptor. by |
| of beta- cell K(ATP) channels | | | inhibiting sulfonylurea from binding to the receptor, it |
| | | | reduces k(atp) channel currents and thereby stimulates |
| | | | insulin secretion |
| Sec61 gamma; necessary for | AF054184 | ER | necessary for protein translocation in the endoplasmic |
| protein translocation in the | | | reticulum |
| endoplasmic reticulum | | | |
| like mouse brain protein E46; | AL050282 | | defects in sca10 are the cause of spinocerebellar ataxia |
| ataxin-10 (spinocerebellar | | | type 10 |
| ataxia type 10 protein) | | | |
| ATP synthase, H+ | D16562 | mitochondrial | |
| transporting, mitochondrial | | inner | |
| F1 complex, gamma | | membrane | |
| polypeptide 1 | | | |
| ATP synthase, H+ | AF047436 | mitochondrial | |
| transporting, mitochondrial | | inner | |
| F0 complex, subunit f, | | membrane | |
| isoform 2 | | | |
| voltage-dependent anion | L06132 | mitochondrial | Porin; |
| channel 1; Outer membrane; | | outer | |
| Porin; Mitochondrion | | membrane | |
| 3-oxoacid CoA transferase; | U62961 | mitochondrial | key enzyme for ketone body catabolism. transfers the |
| Mitochondrion; Transferase | | matrix | coa moiety from succinate to acetoacetate. formation |
| | | | of the enzyme-coa intermediate proceeds via an |
| | | | unstable anhydride species formed between the |
| | | | carboxylate groups of the enzyme and substrate |
| dynamin 1-like; This protein | AF000430 | mitochondrial | This protein |
| establishes mitochondrial | | matrix | establishes mitochondrial morphology through a role in |
| morphology through a role in | | Cytoplasm | distributing |
| distributing mitochondrial | | | mitochondrial tubules throughout the cytoplasm. |
| tubules throughout the | | | |
| cytoplasm. | | | |
| cytochrome c oxidase subunit | NM_001865 | mitochondrial | complex IV |
| VIIa polypeptide 2 (liver) | | inner | |
| | | membrane | |
| macrophage migration | L19686 | | |
| inhibitory factor (MIF) | | | |
| proteasome (prosome, | D78275 | cytoplasmic | involved in the atp-dependent degradation of |
| macropain) 26S subunit, | | and nuclear | ubiquitinated proteins |
| ATPase, 6 | | | |
| Fk506-Binding Protein, Alt. | X52220 | | |
| Splice 2 | | | |

Using a different statistical threshold (p<0.02) an additional two hundred sixty three genes were identified that are differentially expressed in patients having a bipolar disorder. Table 4 provides an inclusive list of the three hundred six genes identified as regulated in patients having bipolar disorder (p level<0.02; fold induction>1.2), their Genebank accession numbers, fold change, and p value.

TABLE 4
Function of Differentially Expressed Genes (p < 0.02)

| Gene Description | Accession # | Fold Change | P value |
|---|---|---|---|
| thymosin, beta 10 | M92383 | −1.31 | 0.01063 |
| Cluster Incl. S81916: phosphoglycerate kinase {alternatively spliced} | S81916 | −1.46 | 0.019787 |
| [human, phosphoglycerate kinase deficient patient with episodes of muscl, | | | |
| mRNA Partial Mutant, 307 nt]/cds = (0,143)//ug = Hs.169313/len = 307 | | | |
| muscle specific gene | AB019392 | −1.53 | 0.002077 |
| reticulon 4 | AB020693 | −1.28 | 0.013635 |
| voltage-dependent anion channel 1 pseudogene | AJ002428 | −1.41 | 0.000727 |
| p21 (CDKN1A)-activated kinase 3 | AF068864 | −1.53 | 0.016696 |
| p21 (CDKN1A)-activated kinase 3 | AF068864 | −1.55 | 0.014761 |
| similar to S. pombe dim1+ | AF023612 | −1.23 | 0.006959 |
| guanine nucleotide binding protein (G protein), alpha 13 | L22075 | 1.49 | 0.018088 |
| tubulin, beta, 2 | X02344 | −1.47 | 0.001895 |
| tubulin, beta, 2 | X02344 | −1.41 | 0.003694 |
| D-dopachrome tautomerase | AF012434 | −1.23 | 0.013399 |
| Cluster Incl. AL050065: Homo sapiens mRNA; cDNA DKFZp566M043 | AL050065 | 1.27 | 0.000158 |
| (from clone DKFZp566M043)/cds = UNKNOWN/gb = AL050065/ | | | |
| gi = 4884295/ug = Hs.212587/len = 1568 | | | |
| keratin, hair, acidic, 3B | X82634 | 1.24 | 0.012361 |
| tyrosine 3-monooxygenase/tryptophan 5-monooxygenase activation protein, | U28964 | −1.39 | 0.007862 |
| zeta polypeptide | | | |
| ribosomal protein S7 | Z25749 | −1.22 | 0.017676 |
| KIAA0316 gene product | AB002314 | −1.37 | 0.013021 |
| fibroblast growth factor 9 (glia-activating factor) | D14838 | −1.29 | 0.014416 |
| Cluster Incl. X95677: H. sapiens mRNA for ArgBPIB protein/ | X95677 | −1.5 | 0.004338 |
| cds = (134,1033)/gb = X95677/gi = 1491701/ug = Hs.169237/len = 2374 | | | |
| KIAA1032 protein | AB028955 | −1.45 | 0.004604 |
| tyrosine 3-monooxygenase/tryptophan 5-monooxygenase activation protein, | X57346 | −1.34 | 0.016325 |
| beta polypeptide | | | |
| pyruvate kinase, muscle | M26252 | −1.22 | 0.012184 |
| dentatorubral-pallidoluysian atrophy (atrophin-1) | U47924 | −1.34 | 0.005434 |
| DNA segment on chromosome 6(unique) 2654 expressed sequence | Y18504 | −1.21 | 0.016847 |
| EGF-like-domain, multiple 4 | AB011541 | −1.3 | 0.008183 |
| acylphosphatase 2, muscle type | X84195 | −1.34 | 0.004579 |
| tachykinin, precursor 1 (substance K, substance P, neurokinin 1, neurokinin 2, | U37529 | −3.12 | 0.011804 |
| neuromedin L, neurokinin alpha, neuropeptide K, neuropeptide gamma) | | | |
| ribosomal protein L10a | AL022721 | −1.28 | 0.017343 |
| gamma-aminobutyric acid (GABA) A receptor, alpha 2 | S62907 | −1.4 | 0.007998 |
| potassium inwardly-rectifying channel, subfamily J, member 6 | U52153 | −1.37 | 0.018175 |
| GNAS complex locus | X04409 | −1.23 | 0.015949 |
| GNAS complex locus | X04409 | −1.28 | 0.004268 |
| somatostatin | AI636761 | −2.74 | 0.006587 |
| RAD51-like 3 (S. cerevisiae) | AF034956 | 1.32 | 0.011514 |
| guanine nucleotide binding protein (G protein), beta 5 | AF017656 | −1.36 | 0.007348 |
| KIAA0377 gene product | AB002375 | −1.22 | 0.014708 |
| ribonuclease H1 | AF039652 | −1.28 | 0.008304 |
| neuropeptide Y | AI198311 | −1.84 | 0.017551 |
| NADH dehydrogenase (ubiquinone) 1 beta subcomplex, 1 (7 kD, MNLL) | AI345944 | −1.39 | 0.004675 |
| FSHD region gene 1 | L76159 | −1.42 | 0.001405 |
| Cluster Incl. AA780435: ae93d06.s1 Homo sapiens cDNA, 3 end/ | AA780435 | 1.25 | 0.015387 |
| clone = 1020491/clone_end = 3/gb = AA780435/gi = 2839766/ug = Hs.204446/ | | | |
| len = 451 | | | |
| T-box, brain, 1 | U49250 | 1.24 | 0.014785 |
| desmocollin 2 | X56807 | 1.22 | 0.005732 |
| amyloid beta (A4) precursor protein-binding, family A, member 2 (X11-like) | AF047348 | −1.32 | 0.007039 |
| Cluster Incl. AL050204: Homo sapiens mRNA; cDNA DKFZp586F1223 | AL050204 | 1.24 | 0.015298 |
| (from clone DKFZp586F1223)/cds = UNKNOWN/gb = AL050204/ | | | |
| gi = 4884443/ug = Hs.28540/len = 1634 | | | |
| chloride intracellular channel 2 | Y12696 | 1.21 | 0.003863 |
| chemokine (C-X3-C) receptor 1 | U20350 | −2.42 | 0.017113 |
| Cluster Incl. AI659108: tu08c09.x1 Homo sapiens cDNA, 3 end/ | AI659108 | −1.28 | 0.016605 |
| clone = IMAGE-2250448/clone_end = 3/gb = AI659108/gi = 4762678/ | | | |
| ug = Hs.99093/len = 492 | | | |
| DKFZP566B183 protein | AL050272 | −1.58 | 0.019681 |
| v-myb myeloblastosis viral oncogene homolog (avian) | M13666 | 1.23 | 0.004209 |
| contactin 1 | Z21488 | −1.79 | 0.004286 |
| chromosome 1 open reading frame 15 | AB007948 | −1.79 | 0.006118 |
| sortilin-related receptor, L(DLR class) A repeats-containing | Y08110 | −1.34 | 0.010094 |
| down-regulator of transcription 1, TBP-binding (negative cofactor 2) | M97388 | −1.24 | 0.003746 |
| vesicle-associated soluble NSF attachment protein receptor (v-SNARE; | AF060902 | −1.28 | 0.003184 |
| homolog of S. cerevisiae VT11) | | | |
| neuronal protein | W28770 | −1.66 | 0.011113 |
| putatative 28 kDa protein | L48692 | −1.36 | 0.019003 |
| Cluster Incl. AL109702: Homo sapiens mRNA full length insert cDNA clone | AL109702 | −1.23 | 0.007009 |
| EUROIMAGE 42138/cds = UNKNOWN/gb = AL109702/gi = 5689811/ | | | |
| ug = Hs.19720/len = 1869 | | | |
| ubiquitin-conjugating enzyme E2M (UBC12 homolog, yeast) | AF075599 | −1.22 | 0.001561 |
| kinesin family member 3B | AB002357 | −1.32 | 0.006715 |
| eukaryotic translation elongation factor 1 alpha 2 | X70940 | −1.34 | 0.015877 |
| RNA 3′-terminal phosphate cyclase | Y11651 | −1.28 | 0.01692 |
| proline-rich Gla (G-carboxyglutamic acid) polypeptide 1 | AF009242 | 1.22 | 0.019808 |
| necdin homolog (mouse) | U35139 | −1.41 | 0.014012 |
| src family associated phosphoprotein 2 | AF051323 | −1.37 | 0.006528 |
| excision repair cross-complementing rodent repair deficiency, | M13194 | −1.21 | 0.003425 |
| complementation group 1 (includes overlapping antisense sequence) | | | |
| Rho guanine nucleotide exchange factor (GEF) 4 | AB029035 | −1.39 | 0.001221 |
| U6 snRNA-associated Sm-like protein LSm7 | AA121509 | −1.32 | 0.010447 |
| glutamate decarboxylase 1 (brain, 67 kD) | M81883 | −1.84 | 0.008965 |
| paraneoplastic antigen MA2 | AB020690 | −1.39 | 0.018583 |
| programmed cell death 6 | AF035606 | −1.33 | 0.004502 |
| cytoplasmic FMRP interacting protein 2 | L47738 | −1.22 | 0.01979 |
| ATP synthase, H+ transporting, mitochondrial F1 complex, delta subunit | AI436567 | −1.27 | 0.002319 |
| transcription elongation factor A (SII)-like 1 | M99701 | −1.2 | 0.016432 |
| Cluster Incl. AL049321: Homo sapiens mRNA; cDNA DKFZp564D156 | AL049321 | 1.27 | 0.019168 |
| (from clone DKFZp564D156)/cds = UNKNOWN/gb = AL049321/ | | | |
| gi = 4500094/ug = Hs.9927/len = 1440 | | | |
| NADH dehydrogenase (ubiquinone) Fe—S protein 4 (18 kD) (NADH- | AA203303 | −1.42 | 0.009172 |
| coenzyme Q reductase) | | | |
| chromosome 14 open reading frame 2 | AF054175 | −1.32 | 0.002134 |
| NADH dehydrogenase (ubiquinone) 1 beta subcomplex, 3 (12 kD, B12) | AA203354 | −1.36 | 0.008742 |
| Cluster Incl. AL031178: Human DNA sequence from clone 341E18 on | AL031178 | −1.68 | 0.007684 |
| chromosome 6p11.2-12.3. Contains a Serine/Threonine Protein Kinase gene | | | |
| (presumptive isolog of a Rat gene) and a novel alternatively spliced gene. | | | |
| Contains a putative CpG island, ESTs and GSSs | | | |
| Cluster Incl. N98670: yy66d08.r1 Homo sapiens cDNA, 5 end/ | N98670 | −1.27 | 0.008167 |
| clone = IMAGE-278511/clone_end = 5/gb = N98670/gi = 1270092/ | | | |
| ug = Hs.111632/len = 574 | | | |
| endosulfine alpha | AI658639 | −1.3 | 0.010107 |
| endosulfine alpha | X99906 | −1.55 | 0.001717 |
| microsomal glutathione S-transferase 3 | AF026977 | −1.39 | 0.001451 |
| proteasome (prosome, macropain) subunit, beta type, 7 | D38048 | −1.28 | 0.004323 |
| non-metastatic cells 1, protein (NM23A) expressed in | AL038662 | −1.65 | 0.008898 |
| DR1-associated protein 1 (negative cofactor 2 alpha) | AI991040 | −1.28 | 0.002766 |
| ADP-ribosylation factor 3 | M74491 | −1.21 | 0.012197 |
| methionine-tRNA synthetase | X94754 | −1.2 | 0.004773 |
| HMT1 hnRNP methyltransferase-like 1 (S. cerevisiae) | X99209 | −1.21 | 0.018481 |
| glypican 3 | U50410 | 1.23 | 0.005816 |
| putative breast adenocarcinoma marker (32 kD) | AF042384 | −1.21 | 0.009768 |
| KIAA0935 protein | AB023152 | −1.24 | 0.009612 |
| microtubule-associated proteins 1A/1B light chain 3 | W28807 | −1.27 | 0.002703 |
| cytochrome c oxidase subunit Vb | M19961 | −1.29 | 0.003535 |
| like mouse brain protein E46 | AL050282 | −1.67 | 0.002917 |
| P311 protein | U30521 | −1.3 | 0.017844 |
| nuclear receptor co-repressor 1 | AF044209 | −1.21 | 0.010503 |
| cullin 1 | U58087 | −1.31 | 0.002505 |
| peroxiredoxin 2 | L19185 | −1.31 | 0.007342 |
| nascent-polypeptide-associated complex alpha polypeptide | AF054187 | −1.24 | 0.013613 |
| polymerase (RNA) II (DNA directed) polypeptide B (140 kD) | X63563 | −1.3 | 0.005797 |
| proteasome (prosome, macropain) 26S subunit, non-ATPase, 4 | U51007 | −1.22 | 0.010387 |
| protein phosphatase 3 (formerly 2B), catalytic subunit, beta isoform | M29551 | −1.45 | 0.013912 |
| (calcineurin A beta) | | | |
| ATPase, Ca++ transporting, cardiac muscle, slow twitch 2 | M23115 | −1.4 | 0.011465 |
| KIAA0090 protein | D42044 | 1.21 | 0.01314 |
| ATP synthase, H+ transporting, mitochondrial F1 complex, alpha subunit, | D14710 | −1.38 | 0.001708 |
| isoform 1, cardiac muscle | | | |
| aldolase C, fructose-bisphosphate | AF054987 | −1.29 | 0.01524 |
| isocitrate dehydrogenase 3 (NAD+) beta | AA522698 | −1.26 | 0.004632 |
| ATP synthase, H+ transporting, mitochondrial F1 complex, gamma | D16562 | −1.46 | 0.004519 |
| polypeptide 1 | | | |
| ATP synthase, H+ transporting, mitochondrial F0 complex, subunit f, isoform 2 | AF047436 | −1.48 | 0.001591 |
| dynactin 3 (p22) | W26651 | −1.25 | 0.014741 |
| solute carrier family 25 (mitochondrial carrier; adenine nucleotide | J03592 | −1.28 | 0.008112 |
| translocator), member 6 | | | |
| transcriptional activator of the c-fos promoter | D54318 | −1.41 | 0.004007 |
| transcriptional activator of the c-fos promoter | U49857 | −1.36 | 0.013221 |
| serologically defined breast cancer antigen 84 | AF091085 | −1.24 | 0.004593 |
| glutamic-oxaloacetic transaminase 2, mitochondrial (aspartate | M22632 | −1.26 | 0.019781 |
| aminotransferase 2) | | | |
| RNA binding motif protein 8A | AL049219 | −1.22 | 0.00522 |
| isoleucine-tRNA synthetase | U04953 | −1.32 | 0.01167 |
| cytochrome c oxidase subunit VIb | T57872 | −1.29 | 0.005962 |
| glycogenin | U31525 | −1.25 | 0.019465 |
| melanoma antigen, family D, 1 | W26633 | −1.41 | 0.005774 |
| 3-oxoacid CoA transferase | U62961 | −1.62 | 0.008802 |
| dynamin 1-like | AF000430 | −1.66 | 0.001584 |
| phosphoglycerate mutase 1 (brain) | J04173 | −1.23 | 0.013865 |
| cytochrome c oxidase subunit Va | M22760 | −1.4 | 0.00763 |
| leucine-rich PPR-motif containing | M92439 | −1.31 | 0.015861 |
| cytochrome c oxidase subunit VIIa polypeptide 2 (liver) | AA978033 | −1.42 | 0.00198 |
| ATX1 antioxidant protein 1 homolog (yeast) | U70660 | −1.23 | 0.009151 |
| v-Ki-ras2 Kirsten rat sarcoma 2 viral oncogene homolog | L00049 | −1.36 | 0.006442 |
| eukaryotic translation initiation factor 3, subunit 2 (beta, 36 kD) | U39067 | −1.21 | 0.014181 |
| NADH dehydrogenase (ubiquinone) 1 beta subcomplex, 8 (19 kD, ASHI) | AI541050 | −1.22 | 0.011804 |
| solute carrier family 25 (mitochondrial carrier; adenine nucleotide | J02966 | −1.53 | 0.003831 |
| translocator), member 4 | | | |
| translocase of inner mitochondrial membrane 17 homolog A (yeast) | X97544 | −1.26 | 0.001239 |
| chromogranin B (secretogranin 1) | Y00064 | −2.09 | 0.011271 |
| lactate dehydrogenase B | X13794 | −1.21 | 0.003571 |
| ATPase, H+ transporting lysosomal (vacuolar proton pump), member M | AA877795 | −1.35 | 0.009456 |
| glutathione peroxidase 4 (phospholipid hydroperoxidase) | X71973 | −1.23 | 0.013006 |
| low molecular mass ubiquinone-binding protein (9.5 kD) | AI540957 | −1.37 | 0.00106 |
| palmitoyl-protein thioesterase 1 (ceroid-lipofuscinosis, neuronal 1, infantile) | U44772 | −1 .39 | 0.00881 |
| nardilysin (N-arginine dibasic convertase) | X93209 | −1.21 | 0.011077 |
| ATP synthase, H+ transporting, mitochondrial F0 complex, subunit c (subunit | U09813 | −1.63 | 0.000557 |
| 9) isoform 3 | | | |
| ceroid-lipofuscinosis, neuronal 3, juvenile (Batten, Spielmeyer-Vogt disease) | AC002544 | −1.24 | 0.006393 |
| CGI-51 protein | AL035398 | −1.26 | 0.001213 |
| seryl-tRNA synthetase | X91257 | −1.4 | 0.008777 |
| melanoma antigen, family D, 2 | Z98046 | −1.23 | 0.007847 |
| ATPase, H+ transporting, lysosomal (vacuolar proton pump), alpha | L09235 | −1.54 | 0.00433 |
| polypeptide, 70 kD, isoform 1 | | | |
| NADH dehydrogenase (ubiquinone) Fe—S protein 3 (30 kD) (NADH- | AF067139 | −1.34 | 0.001686 |
| coenzyme Q reductase) | | | |
| golgi associated, gamma adaptin ear containing, ARF binding protein 2 | AC002400 | −1.45 | 0.005359 |
| GDP dissociation inhibitor 2 | Y13286 | −1.31 | 0.01581 |
| Ras-related GTP-binding protein | U41654 | −1.39 | 0.009322 |
| meningioma expressed antigen 5 (hyaluronidase) | AB014579 | −1.26 | 0.011112 |
| Cluster Incl. AF055023: Homo sapiens clone 24723 mRNA sequence/ | AF055023 | 1.26 | 0.004067 |
| cds = UNKNOWN/gb = AF055023/gi = 3005751/ug = Hs.58220/len = 1834 | | | |
| glutamic-oxaloacetic transaminase 1, soluble (aspartate aminotransferase 1) | M37400 | −1 .61 | 0.008363 |
| COP9 (constitutive photomorphogenic, Arabidopsis, homolog) subunit 3 | AF031647 | −1.26 | 0.009059 |
| ribosomal protein L3 | AL022326 | −1.38 | 0.010345 |
| amyloid beta precursor protein binding protein 1, 59 kD | U50939 | −1.26 | 0.005884 |
| ARP2 actin-related protein 2 homolog (yeast) | AF006082 | −1.5 | 0.002224 |
| succinate dehydrogenase complex, subunit B, iron sulfur (Ip) | U17886 | −1.23 | 0.005271 |
| ATP synthase, H+ transporting, mitochondrial F0 complex, subunit d | AF087135 | −1.67 | 0.001078 |
| Cluster Incl. AA527880: nh86h10.s1 Homo sapiens cDNA, 3 end/ | AA527880 | −1.23 | 0.011144 |
| clone = IMAGE-965443/clone_end = 3/gb = AA527880/gi = 2269949/ | | | |
| ug = Hs.661/len = 568 | | | |
| actin related protein 2/3 complex, subunit 3 (21 kD) | AI525393 | −1.49 | 0.000404 |
| polymerase (RNA) II (DNA directed) polypeptide L (7.6 kD) | N24355 | −1.24 | 0.000766 |
| voltage-dependent anion channel 3 | AF038962 | −1.3 | 0.009254 |
| ubiquinol-cytochrome c reductase hinge protein | AA526497 | −1.37 | 0.002659 |
| ATP synthase, H+ transporting, mitochondrial F0 complex, subunit F6 | AA845575 | −1.39 | 0.003637 |
| proteasome (prosome, macropain) subunit, alpha type, 6 | X5941 7 | −1.37 | 0.003532 |
| dynactin 1 (p150, glued homolog, Drosophila) | AF086947 | −1.23 | 0.016308 |
| protein tyrosine phosphatase, receptor type, N polypeptide 2 | U81561 | −1.41 | 0.019536 |
| cytochrome c oxidase subunit VIc | W51774 | −1.43 | 0.003852 |
| NADH dehydrogenase (ubiquinone) 1 alpha subcomplex, 1 (7.5 kD, MWFE) | N47307 | −1.34 | 0.004606 |
| tubulin-specific chaperone c | U61234 | −1.24 | 0.003964 |
| low density lipoprotein-related protein-associated protein 1 (alpha-2- | M63959 | −1.2 | 0.013075 |
| macroglobulin receptor-associated protein 1) | | | |
| glyoxalase 1 | D13315 | −1.32 | 0.012924 |
| glycyl-tRNA synthetase | U09510 | −1.27 | 0.012995 |
| glycyl-tRNA synthetase | U09510 | −1.32 | 0.011219 |
| aldo-keto reductase family 1, member B1 (aldose reductase) | X15414 | −1.35 | 0.004964 |
| nucleolar and coiled-body phosphprotein 1 | D21262 | −1.24 | 0.002917 |
| cytochrome c oxidase subunit VIIb | N50520 | −1.56 | 0.001375 |
| coatomer protein complex, subunit alpha | U24105 | −1.32 | 0.01774 |
| ATP synthase, H+ transporting, mitochondrial F1 complex, O subunit | X83218 | −1.48 | 0.001619 |
| (oligomycin sensitivity conferring protein) | | | |
| dynein, cytoplasmic, heavy polypeptide 1 | AB002323 | −1.28 | 0.012195 |
| uncharacterized bone marrow protein BM036 | AI057607 | −1.26 | 0.005077 |
| farnesyl diphosphate synthase (farnesyl pyrophosphate synthetase, | D14697 | −1.32 | 0.016932 |
| dimethylallyltranstransferase, geranyltranstransferase) | | | |
| NADH dehydrogenase (ubiquinone) flavoprotein 1 (51 kD) | AF053070 | −1.25 | 0.012356 |
| ATPase, H+ transporting, lysosomal (vacuolar proton pump) 31 kD | X76228 | −1.4 | 0.010499 |
| UDP-glucose pyrophosphorylase 2 | U27460 | −1.44 | 0.001884 |
| ATPase, vacuolar, 14 kD | D49400 | −1.27 | 0.001322 |
| inner membrane protein, mitochondrial (mitofilin) | L42572 | −1.22 | 0.017318 |
| DEAD/H (Asp-Glu-Ala-Asp/His) box polypeptide 1 | X70649 | −1.37 | 0.005672 |
| uroporphyrinogen decarboxylase | AF104421 | −1.29 | 0.005848 |
| complement component 1, q subcomponent binding protein | M69039 | −1.33 | 0.00363 |
| solute carrier family 25 (mitochondrial carrier; phosphate carrier), member 3 | X60036 | −1.35 | 0.008737 |
| Cluster Incl. L08666: Homo sapiens porin (por) mRNA, complete cds and | L08666 | −1.35 | 0.010491 |
| truncated cds/cds = UNKNOWN/gb = L08666/gi = 190199/ug = Hs.78902/ | | | |
| len = 1464 | | | |
| mitochondrial ribosomal protein L3 | X06323 | −1.43 | 0.000177 |
| protein-L-isoaspartate (D-aspartate) O-methyltransferase | D25547 | −1.75 | 0.006481 |
| proteasome (prosome, macropain) 26S subunit, ATPase, 5 | AF035309 | −1.23 | 0.010559 |
| IK cytokine, down-regulator of HLA II | AJ005579 | −1.25 | 0.00818 |
| hepatitis B virus x-interacting protein (9.6 kD) | AF029890 | −1.3 | 0.009123 |
| NADH dehydrogenase (ubiquinone) Fe—S protein 5 (15 kD) (NADH- | AI541336 | −1.27 | 0.012446 |
| coenzyme Q reductase) | | | |
| ATP synthase, H+ transporting, mitochondrial F0 complex, subunit c (subunit | X69907 | −1.28 | 0.004027 |
| 9), isoform 1 | | | |
| cytochrome c oxidase subunit VIII | AI525665 | −1.22 | 0.003607 |
| chromobox homolog 3 (HP1 gamma homolog, Drosophila) | AI740522 | −1.26 | 0.003802 |
| proteasome (prosome, macropain) subunit, alpha type, 1 | M64992 | −1.31 | 0.017706 |
| Cluster Incl. U66042: Human clone 191B7 placenta expressed mRNA from | U66042 | −1.2 | 0.002954 |
| chromosome X/cds = UNKNOWN/gb = U66042/gi = 1519267/ug = Hs.82171/ | | | |
| len = 1327 | | | |
| glutathione synthetase | U34683 | −1.23 | 0.014357 |
| peroxiredoxin 4 | U25182 | −1.28 | 0.014485 |
| Sjogren syndrome antigen B (autoantigen La) | X69804 | −1.22 | 0.01958 |
| hypothetical protein MGC10715 | AL049650 | −1.22 | 0.016112 |
| peptidylglycine alpha-amidating monooxygenase | M37721 | −1.39 | 0.016292 |
| dynactin 2 (p50) | U50733 | −1.23 | 0.013766 |
| single-stranded DNA-binding protein 1 | AA768912 | −1.42 | 0.003143 |
| single-stranded DNA-binding protein 1 | AA768912 | −1.3 | 0.014364 |
| eukaryotic translation initiation factor 4B | X55733 | −1.2 | 0.014218 |
| GCN5 general control of amino-acid synthesis 5-like 1 (yeast) | AI525379 | −1.37 | 0.001552 |
| nitrogen fixation cluster-like | U47101 | −1.29 | 0.018877 |
| Sec61 gamma | AF054184 | −1.39 | 0.000911 |
| transcription elongation factor B (SIII), polypeptide 2 (18 kD, elongin B) | AI857469 | −1.25 | 0.004063 |
| ectonucleoside triphosphate diphosphohydrolase 6 (putative function) | AL035252 | −1.24 | 0.00585 |
| cutaneous T-cell lymphoma-associated tumor antigen se20-4; differentially | AB015345 | −1.28 | 0.010929 |
| expressed nucleolar TGF-beta 1 target protein (DENTT) | | | |
| SET translocation (myeloid leukemia-associated) | M93651 | −1.27 | 0.007058 |
| voltage-dependent anion channel 1 | L06132 | −1.49 | 0.00374 |
| NADH dehydrogenase (ubiquinone) 1 alpha subcomplex, 2 (8 kD, B8) | AF047185 | −1.27 | 0.002062 |
| eukaryotic translation elongation factor 1 epsilon 1 | AF054186 | −1.33 | 0.017208 |
| hypothetical protein | H15872 | −1.27 | 0.011372 |
| Cluster Incl. AI382123: te30a09.x1 Homo sapiens cDNA, 3 end/ | AI382123 | −1.43 | 0.01227 |
| clone = IMAGE-2087416/clone_end = 3/gb = AI382123/gi = 4194904/ | | | |
| ug = Hs.182919/len = 857 | | | |
| SWI/SNF related, matrix associated, actin dependent regulator of chromatin, | D26155 | −1.36 | 0.002574 |
| subfamily a, member 2 | | | |
| KIAA0447 gene product | AB007916 | −1.22 | 0.018871 |
| JTV1 gene | U24169 | −1.23 | 0.01197 |
| thyroid hormone receptor interactor 3 | L40410 | −1.31 | 0.007183 |
| KIAA1049 protein | AB028972 | −1.37 | 0.003695 |
| integral membrane protein 2B | AA477898 | −1.32 | 0.008173 |
| lactate dehydrogenase A | X02152 | −1.37 | 0.009983 |
| protein phosphatase 1, regulatory subunit 7 | Z50749 | −1.36 | 0.001411 |
| adaptor-related protein complex 1, sigma 2 subunit | AF091077 | −1.38 | 0.015644 |
| Cluster Incl. AA203545: zx59a05.r1 Homo sapiens cDNA, 5 end/ | AA203545 | −1.29 | 0.018083 |
| clone = IMAGE-446768/clone_end = 5/gb = AA203545/gi = 1799271/ | | | |
| ug = Hs.56876/len = 568 | | | |
| emopamil binding protein (sterol isomerase) | Z37986 | −1.2 | 0.013307 |
| fumarate hydratase | U59309 | −1.47 | 0.003497 |
| protein translocation complex beta | AA083129 | −1.21 | 0.009925 |
| proteasome (prosome, macropain) 26S subunit, non-ATPase, 8 | D38047 | −1.3 | 0.014744 |
| regulator of G-protein signalling 10 | AF045229 | −1.3 | 0.002964 |
| UDP-GlcNAc: betaGal beta-1,3-N-acetylglucosaminyltransferase 6 | AF029893 | −1.5 | 0.003738 |
| proteasome (prosome, macropain) subunit, beta type, 4 | D26600 | −1.39 | 0.004463 |
| ras-related C3 botulinum toxin substrate 1 (rho family, small GTP binding | M29870 | −1.26 | 0.012621 |
| protein Rac1) | | | |
| APEX nuclease (multifunctional DNA repair enzyme) | M80261 | −1.2 | 0.007584 |
| S-phase kinase-associated protein 1A (p19A) | U33760 | −1.42 | 0.002275 |
| non-metastatic cells 1, protein (NM23A) expressed in | X73066 | −1.23 | 0.010548 |
| RAN, member RAS oncogene family | M31469 | −1.4 | 0.007467 |
| COP9 (constitutive photomorphogenic, Arabidopsis, homolog) subunit 5 | U65928 | −1.38 | 0.002999 |
| platelet-derived growth factor receptor, alpha polypeptide | M21574 | 1.27 | 0.007996 |
| mitogen-activated protein kinase 10 | U07620 | −1.21 | 0.00607 |
| neural precursor cell expressed, developmentally down-regulated 8 | D23662 | −1.23 | 0.011055 |
| Ras homolog enriched in brain 2 | D78132 | −1.2 | 0.004309 |
| ubiquitin-conjugating enzyme E2N (UBC13 homolog, yeast) | D83004 | −1.33 | 0.003258 |
| RAP1, GTP-GDP dissociation stimulator 1 | X63465 | −1.54 | 0.009557 |
| melanoma-associated antigen recognised by cytotoxic T lymphocytes | U19796 | −1.22 | 0.010698 |
| U50535/FEATURE = /DEFINITION = HSU50535 Human BRCA2 region, | U50535 | 1.23 | 0.009684 |
| mRNA sequence CG006 | | | |
| protein tyrosine phosphatase, receptor type, A | M34668 | −1.25 | 0.015648 |
| heat shock protein 75 | U12595 | −1.31 | 0.005772 |
| proteasome (prosome, macropain) subunit, alpha type, 2 | D00760 | −1.3 | 0.008858 |
| proteasome (prosome, macropain) subunit, alpha type, 3 | D00762 | −1.39 | 0.012143 |
| somatostatin | J00306 | −1.31 | 0.012147 |
| transcription elongation factor B (SIII), polypeptide 1 (15 kD, elongin C) | L34587 | −1.32 | 0.00108 |
| replication protein A1 (70 kD) | M63488 | −1.25 | 0.01319 |
| X14675/FEATURE = cds/DEFINITION = HSBCR3C Human bcr-abl mRNA | X14675 | 1.27 | 0.011268 |
| 5 fragment (clone 3c) | | | |
| retinoblastoma binding protein 4 | X74262 | −1.21 | 0.013161 |
| proteasome (prosome, macropain) subunit, beta type, 3 | D26598 | −1.24 | 0.000943 |
| proteasome (prosome, macropain) subunit, beta type, 2 | D26599 | −1.31 | 0.005831 |
| proteasome (prosome, macropain) subunit, beta type, 4 | D26600 | −1.33 | 0.001273 |
| proteasome (prosome, macropain) 26S subunit, non-ATPase, 8 | D38047 | −1.3 | 0.004844 |
| proteasome (prosome, macropain) subunit, beta type, 7 | D38048 | −1.35 | 0.002867 |
| proteasome (prosome, macropain) 26S subunit, non-ATPase, 1 | D44466 | −1.36 | 0.001895 |
| tyrosine 3-monooxygenase/tryptophan 5-monooxygenase activation protein, | M86400 | −1.47 | 0.010563 |
| zeta polypeptide | | | |
| cyclin-dependent kinase 5 | X66364 | −1.27 | 0.017644 |
| proteasome (prosome, macropain) 26S subunit, non-ATPase, 11 | AB003102 | −1.36 | 0.005788 |
| neuregulin 1 | L12260 | 1.21 | 0.009292 |
| histidine triad nucleotide binding protein | U51004 | −1.29 | 0.009004 |
| proteasome (prosome, macropain) 26S subunit, ATPase, 6 | D78275 | −1.49 | 0.002007 |
| Fk506-Binding Protein, Alt. Splice 2 | X52220 | −1.32 | 0.013334 |
| glycosylphosphatidylinositol specific phospholipase D1 | L11702 | 1.2 | 0.000988 |
| macrophage migration inhibitory factor (glycosylation-inhibiting factor) | L19686 | −1.36 | 0.000667 |
| FK506 binding protein 1A (12 kD) | M34539 | −1.25 | 0.008632 |
| ubiquitin carrier protein | M91670 | −1.28 | 0.014471 |
| glutathione-S-transferase like; glutathione transferase omega | U90313 | −1.3 | 0.015862 |
| v-crk sarcoma virus CT10 oncogene homolog (avian) | D10656 | −1.24 | 0.018048 |
| GDP dissociation inhibitor 2 | D13988 | −1.24 | 0.008673 |
| protease, serine, 11 (IGF binding) | D87258 | −1.21 | 0.019954 |
| proteasome (prosome, macropain) 26S subunit, ATPase, 1 | L02426 | −1.21 | 0.010531 |
| RAB5A, member RAS oncogene family | M28215 | −1.24 | 0.013948 |
| proteasome (prosome, macropain) 26S subunit, ATPase, 3 | M34079 | −1.26 | 0.001283 |
| polymerase (RNA) II (DNA directed) polypeptide L (7.6 kD) | U37690 | −1.27 | 0.000267 |
| tubulin, beta, 4 | U47634 | −1.38 | 0.008012 |
| tubulin, beta, 5 | X00734 | −1.37 | 0.008912 |
| casein kinase 2, beta polypeptide | X57152 | −1.24 | 0.013439 |
| dynamin 1-like | AF000430 | −1.3 | 0.009226 |
| basic transcription factor 3 | X53280 | −1.2 | 0.017502 |
| tubulin, alpha 1 (testis specific) | X06956 | −1.67 | 0.011897 |
| microtubule-associated protein tau | J03778 | −1.31 | 0.002685 |
| ubiquinol-cytochrome c reductase core protein 1 | L16842 | −1.4 | 0.004071 |
| H2A histone family, member O | L19779 | −1.22 | 0.012995 |
| calcium/calmodulin-dependent protein kinase 1 | L41816 | −1.29 | 0.007763 |
| S-adenosylmethionine decarboxylase 1 | M21154 | −1.28 | 0.019389 |
| protein kinase, cAMP-dependent, regulatory, type 1, alpha (tissue specific | M33336 | −1.3 | 0.012561 |
| extinguisher 1) | | | |
| protein kinase, cAMP-dependent, regulatory, type 1, alpha (tissue specific | M33336 | −1.33 | 0.013602 |
| extinguisher 1) | | | |
| IK cytokine, down-regulator of HLA II | S74221 | −1.23 | 0.012642 |
| ubiquitin-conjugating enzyme E2L 3 | S81003 | −1.29 | 0.011308 |
| aconitase 2, mitochondrial | U87939 | −1.26 | 0.011241 |

Expression Regulation among Gene Families

To further explore regulation trends in gene families, the MAPPfinder computer program (Dahlquist et al., Nature Genetics 31:19-20, 2002) was used. To further explore regulation trends in gene families, the MAPPfinder computer program (Dahlquist et al., Nature Genetics 31:19-20, 2002) was used to compare all genes found to be downregulated in bipolar disorder to the databank of the Gene Ontology Consortium (publicly available at the consortium's website). This program groups genes into “terms” by molecular function, biological process, or cellular component. A ranking was obtained of all the gene ontology terms met by the samples, and a corresponding z score, was returned. Of the six groups with a z score above 10, three groups were associated with mitochondrial energy production and three were associated with ATP-dependent proteasomal degradation (Table 5).

TABLE 5
Gene Ontology Groups

| Gene Ontology Name | GO ID No. | Type | Changed/Measured/Present | z Score |
|---|---|---|---|---|
| Mitochondrion | | | | |
| Mitochondrial membrane | 5740 | Cellular component | 20/75/189 | 11.4 |
| Mitochondrial inner membrane | 5743/19866 | Cellular component | 17/50/154 | 12.2 |
| Proton-transporting ATP synthase complex | 5753 | Cellular component | 7/9/13 | 12.5 |
| Proteasome | | | | |
| 26S Proteasome | 5837 | Cellular component | 14/32/43 | 12.8 |
| 20S Proteasome | 5839 | Cellular component | 8/16/23 | 10.4 |
| Threonine endopeptidase | 4299 | Molecular Function | 8/13/20 | 11.7 |

*GO ID NO. = gene ontology identification number

Of those genes encoding proteins that function in the mitochondria, MAPPfinder identified 50 mRNAs that encode proteins located in the mitochondrial inner membrane. The expression of seventeen of these genes (34%) was decreased in bipolar disorder. Furthermore, a decrease in the expression of seven of nine (78%) genes associated with the ATP synthase complex, which functions in proton-transport in the inner mitochondrial membrane, was observed in hippocampi from patients with bipolar disorder.

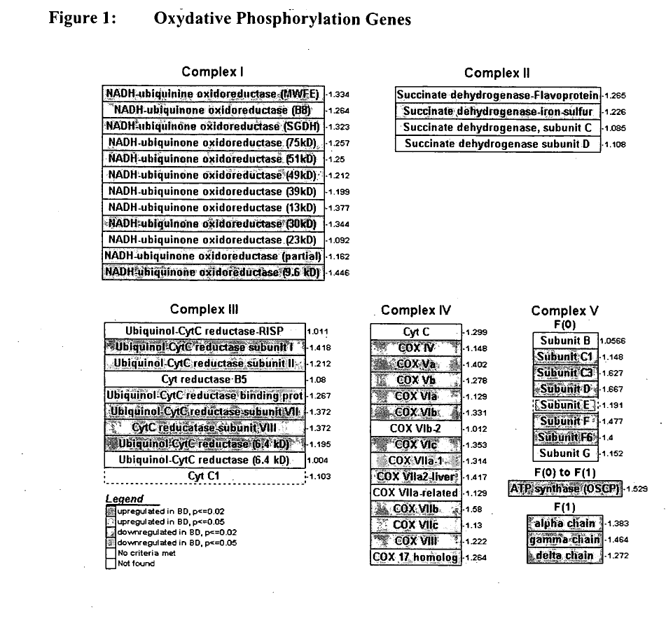

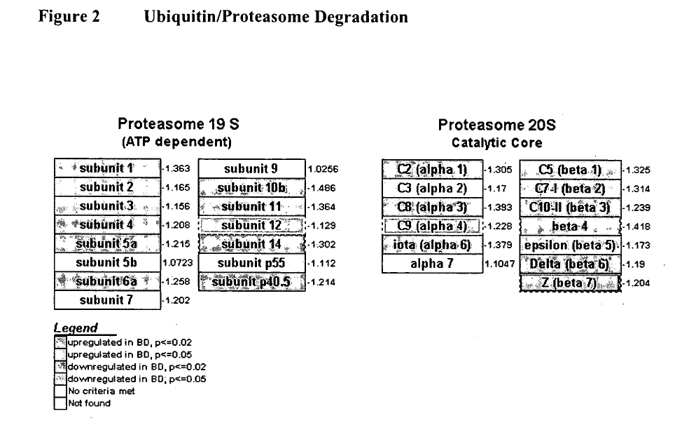

To further investigate the regulation of genes involved in energy metabolism and proteasome degradation, maps of the enzymes involved in oxidative phosphorylation (FIG. 1) and proteasome degradation (FIG. 2) were drawn using GenMAPP (Dahlquist et al., Nature Genetics. 31:19-20, 2002). These gene maps revealed that gene expression decreases observed in bipolar disorder were pronounced and widespread. Mapping analysis of these genes in schizophrenia identified no significant differences from the normal group. Not one of the genes encoding sixty-two proteins involved in oxidative phosphorylation or twenty-eight proteins involved in proteasome degradation was decreased. This demonstrated that the observed changes in gene expression were specific to bipolar disorder.

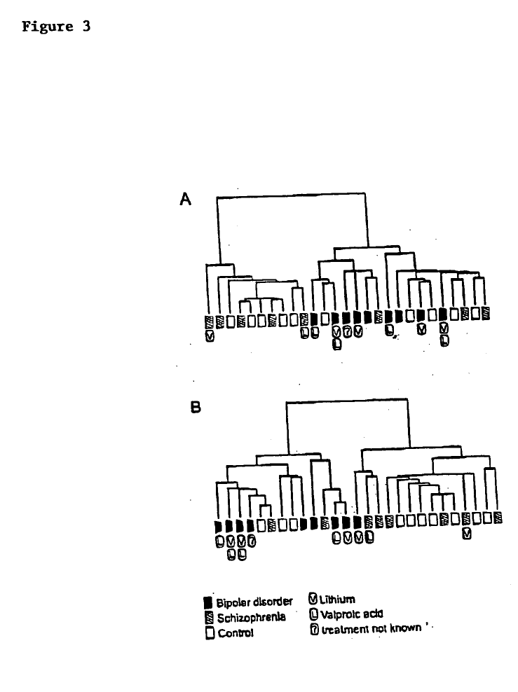

We performed hierarchical clustering using dChip to identify samples with similar expression profiles (FIGS. 3A and 3B). To limit noise and increase strength of findings, only genes with ample variability and ‘present’ calls were used for clustering. Variability was set to a standard deviation above 4% of the mean of the expression value, and genes had to be deemed ‘present’ in at least 20% of samples. A total of 216 genes met the criteria. These genes showed that bipolar disorder samples had similar genetic profiles and clustered together (p=0.005).

Quantitative Real-Time PCR

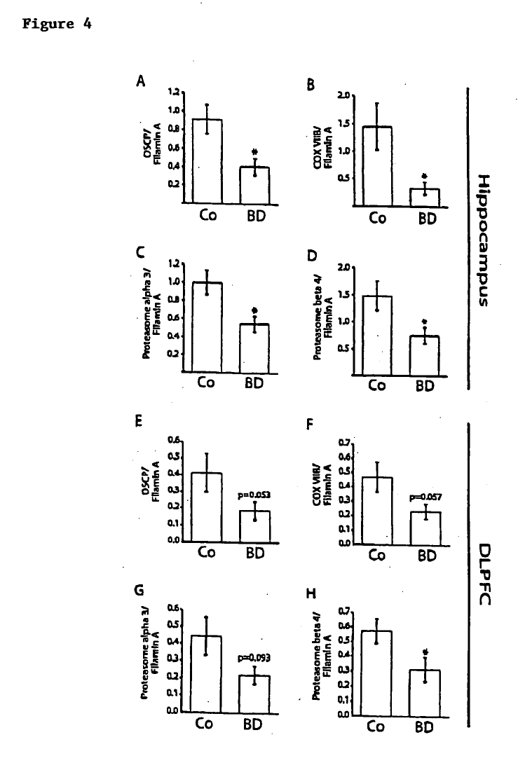

Observations regarding gene regulation were confirmed for five exemplary genes, oligomycin sensitivity conferring protein (OSCP), a subunit of the mitochondrial ATP synthase, B, the mitochondrial cytochrome c oxidase subunit COX VIIb, C, the proteasome alpha 3 subunit, D, the proteasome beta 4 subunit, and human ubiquinol cytochrome-c reductase core I protein mRNA, using quantitative real-time PCR (Q-rt-PCR). All five mRNAs were corrected for an internal control gene (filamin A), and were significantly downregulated, confirming the gene array data. The results for four of these genes are shown in FIGS. 4A and 4B.

To explore whether the decreased expression was specific for the hippocampus, we performed the same Q-rt-PCR analysis in frontal cortex specimens of the same cases. These results for four genes are shown in FIGS. 4A and 4B. We found a similar pattern of decreased expression in the frontal cortex tissue in bipolar disorder subjects.

For Q-rt-PCR, cDNA was synthesized from 1 μg of total RNA with the Invitrogen SuperScript First-Strand Synthesis System for Q-rt-PCR (Invitrogen, CA), using Oligo dT as the Primer. A primer set for each gene was designed with the help of Primer3 (www-genome.wi.mit.edu/cgi-bin/primer/primer3.cgi). Amplicons were designed to be between 100 and 150 base pairs in length. Melt curve analysis and polyacrylamide gel electrophoresis were used to confirm the specificity of each primer pair. The real-time QQ-rt-PCR reaction was performed in the MJ RESEARCH DNA ENGINE OPTICON (MJ Research, Waltham, Mass.; Opticon Monitor Data Analysis Software v 1.4), with the DyNAmo SYBR Green Q-rt-PCR Kit (Finnzymes, Finland), according to the company protocol, in 25 μl volume, with 2.5 μl of 1:5 diluted cDNA samples and 0.3 μM Primers. PCR cycling conditions were as follows: initially, samples were heated at 95° C. for 10 minutes, followed by 49 cycles of 94° C. for 30 seconds, 55° C. for 30 seconds, 72° C. for 30 seconds. Data were collected between 72° C. and 79° C., depending on amplicon melting temperature. A melt curve analysis was performed at the end of each Q-rt-PCR experiment. Dilution curves were generated for each primer in every experiment by diluting cDNA from a control sample 1:3 twice, yielding a dilution series of 1.00, 0.333, and 0.111. The log of the dilution value was plotted against the cycle threshold (CT) value. Blanks were run with each dilution curve to control for cross contamination. Dilution curves, blanks, and samples were run in duplicate. Reported values were normalized to the internal control Human Filamin A alpha (accession # NM_{—}001456), an actin binding protein. Filamin A alpha was not regulated in the gene array analysis or in the Q-rt-PCR analysis. Seven control samples and six bipolar disorder samples, available from the original group, were used for Q-rt-PCR.

The identical Q-rt-PCR parameters were used for an analysis of 16 frontal lobe specimens (8 control and 8 bipolar subjects of the original study sample). Cortical tissue was removed from Brodmann area 9 and RNA was extracted as detailed above.

The present invention provides for nucleic acid or polypeptide compositions that can be employed in an array-format for detecting changes in expression of a number of genes that are differentially regulated in bipolar disorder. Microarrays are useful in the diagnosis of a bipolar disorder and in monitoring treatments where altered expression of nuclear encoded mitochondrial energy metabolism polypeptides is associated with a bipolar disorder. In addition, the microarrays can also be used to investigate an individual's predisposition to a bipolar disorder.

Microarrays

The nucleic acid molecules or polypeptides of the invention are useful as hybridizable array elements in a microarray. The array elements are organized in an ordered fashion such that each element is present at a specified location on the substrate. Useful substrate materials include membranes, composed of paper, nylon or other materials, filters, chips, glass slides, and other solid supports. The ordered arrangement of the array elements allows hybridization patterns and intensities to be interpreted as expression levels of particular genes or proteins. Methods for making nucleic acid microarrays are known to the skilled artisan and are described, for example, in U.S. Pat. No. 5,837,832, Lockhart, et al. (Nat. Biotech. 14:1675-1680, 1996), and Schena, et al. (Proc. Natl. Acad. Sci. 93:10614-10619, 1996), herein incorporated by reference. Methods for making polypeptide microarrays are described, for example, by Ge (Nucleic Acids Res. 28: e3. i-e3. vii, 2000), MacBeath et al., (Science 289:1760-1763, 2000), Zhu et al.(Nature Genet. 26:283-289), and in U.S. Pat. No. 6,436,665, hereby incorporated by reference.

Nucleic Acid Microarrays

To produce a nucleic acid microarray oligonucleotides may be synthesized or bound to the surface of a substrate using a chemical coupling procedure and an ink jet application apparatus, as described in PCT application W095/251116 (Baldeschweiler et al.), incorporated herein by reference. Alternatively, a gridded array may be used to arrange and link cDNA fragments or oligonucleotides to the surface of a substrate using a vacuum system, thermal, UV, mechanical or chemical bonding procedure.

A nucleic acid molecule (e.g. RNA or DNA) derived from a biological sample may be used to produce a hybridization probe as described herein. The biological samples are generally derived from a patient, preferably as a bodily fluid (such as blood, cerebrospinal fluid, phlegm, saliva, or urine) or tissue sample (e.g. a tissue sample obtained by biopsy). For some applications, cultured cells (e.g., lymphocytes) or other tissue preparations may be used. The mRNA is isolated according to standard methods, and cDNA is produced and used as a template to make complementary RNA suitable for hybridization. Such methods are described herein. The RNA is amplified in the presence of fluorescent nucleotides, and the labeled probes are then incubated with the microarray to allow the probe sequence to hybridize to complementary oligonucleotides bound to the microarray.

Incubation conditions are adjusted such that hybridization occurs with precise complementary matches or with various degrees of less complementarity depending on the degree of stringency employed. For example, stringent salt concentration will ordinarily be less than about 750 mM NaCl and 75 mM trisodium citrate, preferably less than about 500 mM NaCl and 50 mM trisodium citrate, and most preferably less than about 250 mM NaCl and 25 mM trisodium citrate. Low stringency hybridization can be obtained in the absence of organic solvent, e.g., formamide, while high stringency hybridization can be obtained in the presence of at least about 35% formamide, and most preferably at least about 50% formamide. Stringent temperature conditions will ordinarily include temperatures of at least about 30° C., more preferably of at least about 37° C., and most preferably of at least about 42° C. Varying additional parameters, such as hybridization time, the concentration of detergent, e.g., sodium dodecyl sulfate (SDS), and the inclusion or exclusion of carrier DNA, are well known to those skilled in the art. Various levels of stringency are accomplished by combining these various conditions as needed. In a preferred embodiment, hybridization will occur at 30° C. in 750 mM NaCl, 75 mM trisodium citrate, and 1% SDS. In a more preferred embodiment, hybridization will occur at 37° C. in 500 mM NaCl, 50 mM trisodium citrate, 1% SDS, 35% formamide, and 100 μg/ml denatured salmon sperm DNA (ssDNA). In a most preferred embodiment, hybridization will occur at 42° C. in 250 mM NaCl, 25 mM trisodium citrate, 1% SDS, 50% formamide, and 200 μg/ml ssDNA. Useful variations on these conditions will be readily apparent to those skilled in the art.

The removal of nonhybridized probes may be accomplished, for example, by washing. The washing steps that follow hybridization can also vary in stringency. Wash stringency conditions can be defined by salt concentration and by temperature. As above, wash stringency can be increased by decreasing salt concentration or by increasing temperature. For example, stringent salt concentration for the wash steps will preferably be less than about 30 mM NaCl and 3 mM trisodium citrate, and most preferably less than about 15 mM NaCl and 1.5 mM trisodium citrate. Stringent temperature conditions for the wash steps will ordinarily include a temperature of at least about 25° C., more preferably of at least about 42° C., and most preferably of at least about 68° C. In a preferred embodiment, wash steps will occur at 25° C. in 30 mM NaCl, 3 mM trisodium citrate, and 0.1% SDS. In a more preferred embodiment, wash steps will occur at 42° C. in 15 mM NaCl, 1.5 mM trisodium citrate, and 0.1% SDS. In a most preferred embodiment, wash steps will occur at 68° C. in 15 mM NaCl, 1.5 mM trisodium citrate, and 0.1% SDS. Additional variations on these conditions will be readily apparent to those skilled in the art.

A detection system may be used to measure the absence, presence, and amount of hybridization for all of the distinct sequences simultaneously (e.g., Heller et al., Proc. Natl. Acad. Sci. 94:2150-2155, 1997). Preferably, a scanner is used to determine the levels and patterns of fluorescence.

Protein Microarrays

Families of proteins, such as those described herein, may also be analyzed using protein microarrays. Such arrays are useful in high-throughput low-cost screens to identify peptide or candidate compounds that bind a polypeptide of the invention, or fragment thereof. Typically, protein microarrays feature a protein, or fragment thereof, bound to a solid support. Suitable solid supports include membranes (e.g., membranes composed of nitrocellulose, paper, or other material), polymer-based films (e.g., polystyrene), beads, or glass slides. For some applications, proteins (e.g., polypeptides encoded by a nucleic acid molecule listed in Table 2 or Table 4 or antibodies against such polypeptides) are spotted on a substrate using any convenient method known to the skilled artisan (e.g., by hand or by inkjet printer). Preferably, such methods retain the biological activity or function of the protein bound to the substrate (Ge et al., supra; Zhu et al., supra).

The protein microarray is hybridized with a detectable probe. Such probes can be polypeptide, nucleic acid, or small molecules. For some applications, polypeptide and nucleic acid probes are derived from a biological sample taken from a patient, such as a bodily fluid (such as blood, urine, saliva, or phlegm); a homogenized tissue sample (e.g. a tissue sample obtained by biopsy); or cultured cells (e.g., lymphocytes). Probes can also include antibodies, candidate peptides, nucleic acids, or small molecule compounds derived from a peptide, nucleic acid, or chemical library. Hybridization conditions (e.g., temperature, pH, protein concentration, and ionic strength) are optimized to promote specific interactions. Such conditions are known to the skilled artisan and are described, for example, in Harlow, E. and Lane, D., Using Antibodies : A Laboratory Manual. 1998, New York: Cold Spring Harbor Laboratories. After removal of non-specific probes, specifically bound probes are detected, for example, by fluorescence, enzyme activity (e.g., an enzyme-linked calorimetric assay), direct immunoassay, radiometric assay, or any other suitable detectable method known to the skilled artisan.

Diagnostics

Expression levels of particular nucleic acids or polypeptides may be correlated with a particular disease state, and thus are useful in diagnosis.

In one embodiment, a patient having a bipolar disorder will show an alteration in the expression of at least one of the nucleic acids listed in Table 2 or Table 4. In another embodiment, a patient having a bipolar disorder will have a particular expression profile that includes significantly decreased expression of two or more nuclear encoded mitochondrial metabolism nucleic acid molecules or proteasome associated nucleic acid molecules (e.g., those listed in Table 2 or Table 4). Alterations in gene expression are detected using methods known to the skilled artisan and described herein.

In one embodiment, oligonucleotides or longer fragments derived from any of the nucleic acid sequences described herein (e.g., those listed in Table 2 or Table 4) may be used as targets in a microarray. The microarray is used to assay the expression level of large numbers of genes simultaneously and to identify genetic variants, mutations, and polymorphisms. Such information can be used to diagnose a bipolar disorder.

In another embodiment, an alteration in the expression of a nucleic acid sequence described herein (e.g., those listed in Table 2 or Table 4) is detected using real-time quantitative PCR (Q-rt-PCR) to detect changes in gene expression. Q-rt-PCR methods are known in the art and are described herein.

In another embodiment, an antibody that specifically binds a polypeptides encoded by a nucleic acid described herein (e.g., listed in Table 2 or Table 4) may be used for the diagnosis of a bipolar disorder. A variety of protocols for measuring an alteration in the expression of such polypeptides are known, including immunological methods (such as ELISAs and RIAs), and provide a basis for diagnosing a bipolar disorder. Again, a decrease in the level of the polypeptide is diagnostic of a patient having a bipolar disorder.

In yet another embodiment, hybridization with PCR probes that are capable of detecting at least one of the polynucleotide sequences listed in Table 2 or Table 4, including genomic sequences, or closely related molecules, may be used to hybridize to a nucleic acid sequence derived from a patient having a bipolar disorder. The specificity of the probe, whether it is made from a highly specific region, e.g., the 5′ regulatory region, or from a less specific region, e.g., a conserved motif, and the stringency of the hybridization or amplification (maximal, high, intermediate, or low), determine whether the probe hybridizes to a naturally occurring sequence, allelic variants, or other related sequences. Hybridization techniques may be used to identify mutations indicative of a bipolar disorder in genes listed in Table 2 or Table 4, or may be used to monitor expression levels of these genes (for example, by Northern analysis (Ausubel et al., supra).

In yet another approach, humans may be diagnosed for a propensity to develop a bipolar disorder by direct analysis of the sequence of at least one of the nucleic acids listed in Table 2 or Table 4.

Screening Assays

As discussed above, the expression of genes listed in Table 2 is decreased in a bipolar disorder, while the expression of genes listed in Table 4 is altered. Based on these discoveries, compositions of the invention are useful for the high-throughput low-cost screening of candidate compounds to identify those that modulate the expression of a nuclear encoded mitochondrial energy metabolism polypeptide, proteasomal polypeptide, or other polypeptide whose expression is altered in a patient having a bipolar disorder. In one embodiment, the effects of known therapeutic drugs on the expression of a nuclear encoded mitochondrial energy metabolism or proteasomal pathway can be assayed using microarrays of the invention. Tissues or cells treated with these drugs are compared to untreated corresponding control samples to produce expression profiles of known therapeutic agents. Knowing the identity of sequences that are differentially regulated in the presence and absence of a therapeutic agent is useful in understanding the mechanisms of drug action.

Any number of methods are available for carrying out screening assays to identify new candidate compounds that promote the expression of a nuclear encoded mitochondrial pathway or proteasomal pathway component. In one working example, candidate compounds are added at varying concentrations to the culture medium of cultured cells expressing one of the nucleic acid sequences of the invention. Gene expression is then measured, for example, by microarray analysis, Northern blot analysis (Ausubel et al., supra), reverse transcriptase PCR, or quantitative real-time PCR using any appropriate fragment prepared from the nucleic acid molecule as a hybridization probe. The level of gene expression in the presence of the candidate compound is compared to the level measured in a control culture medium lacking the candidate molecule. A compound which promotes an increase in the expression of a nuclear encoded mitochondrial energy metabolism gene, a proteasomal nucleic acid molecule, or any nucleic acid molecule listed in Table 2 or Table 4, or a functional equivalent thereof, is considered useful in the invention; such a molecule may be used, for example, as a therapeutic to treat a bipolar disorder in a human patient.

In another working example, the effect of candidate compounds may be measured at the level of polypeptide production using the same general approach and standard immunological techniques, such as Western blotting or immunoprecipitation with an antibody specific for a polypeptide encoded by a nuclear encoded mitochondrial energy metabolism gene, a proteasomal gene, or any other gene listed in Table 2 or Table 4. For example, immunoassays may be used to detect or monitor the expression of at least one of the polypeptides of the invention in an organism. Polyclonal or monoclonal antibodies, (produced as described above) that are capable of binding to such a polypeptide may be used in any standard immunoassay format (e.g., ELISA, Western blot, or RIA assay) to measure the level of the polypeptide. In some embodiments, a compound that promotes an increase in the expression or biological activity of the polypeptide is considered particularly useful. Again, such a molecule may be used, for example, as a therapeutic to delay, ameliorate, or treat a bipolar disorder, or the symptoms of a bipolar disorder, in a human patient.

In yet another working example, candidate compounds may be screened for those that specifically bind to a polypeptide encoded by a nuclear encoded mitochondrial energy metabolism gene, a proteasomal gene, or any other gene listed in Table 2 or Table 4. The efficacy of such a candidate compound is dependent upon its ability to interact with such a polypeptide or a functional equivalent thereof. Such an interaction can be readily assayed using any number of standard binding techniques and functional assays (e.g., those described in Ausubel et al., supra). In one embodiment, a candidate compound may be tested in vitro for its ability to specifically bind a polypeptide of the invention In another embodiment, a candidate compound is tested for its ability to enhance the biological activity of a polypeptide described herein, such as a nuclear encoded mitochondrial energy metabolism polypeptide or a proteasomal polypeptide. The biological activity of a nuclear encoded mitochondrial energy metabolism polypeptide may be assayed using any standard method, for example, ATP production may be assayed using an adenosine 5-triphosphate (ATP) bioluminescent somatic cell assay kit (Sigma, catalog no. FL-ASC). In another example, the biological activity of a proteasomal polypeptide may be assayed, for example, using a commercially available kit from Affiniti Research Products that monitors the cleavage of a fluorogenic peptide substrate by the 20S proteasome.

In another working example, a nucleic acid described herein (e.g., a nuclear encoded mitochondrial energy metabolism nucleic acid, proteasomal nucleic acid, or any other nucleic acid listed in Table 2 or Table 4) is expressed as a transcriptional or translational fusion with a detectable reporter, and expressed in an isolated cell (e.g., mammalian or insect cell) under the control of a heterologous promoter, such as an inducible promoter. The cell expressing the fusion protein is then contacted with a candidate compound, and the expression of the detectable reporter in that cell is compared to the expression of the detectable reporter in an untreated control cell. A candidate compound that alters (e.g., increases or decreases) the expression of the detectable reporter is a compound that is useful for the treatment of a bipolar disorder. In preferred embodiments, the candidate compound increases the expression of a reporter gene fused to a nuclear encoded mitochondrial energy metabolism nucleic acid or a proteasomal nucleic acid.

In one particular working example, a candidate compound that binds to a polypeptide encoded by a nuclear mitochondrial energy metabolism gene, a proteasomal gene, or any other nucleic acid molecule listed in Table 2 or Table 4 may be identified using a chromatography-based technique. For example, a recombinant polypeptide of the invention may be purified by standard techniques from cells engineered to express the polypeptide (e.g., those described above) and may be immobilized on a column. A solution of candidate compounds is then passed through the column, and a compound specific for the nuclear encoded mitochondrial energy metabolism polypeptide, a proteasomal polypeptide, or any other polypeptide encoded by a nucleic acid molecule listed in Table 2 or Table 4 is identified on the basis of its ability to bind to the polypeptide and be immobilized on the column. To isolate the compound, the column is washed to remove non-specifically bound molecules, and the compound of interest is then released from the column and collected. Similar methods may be used to isolate a compound bound to a polypeptide microarray. Compounds isolated by this method (or any other appropriate method) may, if desired, be further purified (e.g., by high performance liquid chromatography). In addition, these candidate compounds may be tested for their ability to increase the activity of a mitochondrial energy metabolism or proteasomal pathway (e.g., as described herein). Compounds isolated by this approach may also be used, for example, as therapeutics to treat a bipolar disorder in a human patient. Compounds that are identified as binding to a polypeptide of the invention with an affinity constant less than or equal to 10 mM are considered particularly useful in the invention. Alternatively, any in vivo protein interaction detection system, for example, any two-hybrid assay may be utilized.

Potential agonists and antagonists include organic molecules, peptides, peptide mimetics, polypeptides, nucleic acids, and antibodies that bind to a nucleic acid sequence or polypeptide of the invention.(e.g., those listed in Table 2 or Table 4). For those nucleic acid sequences or polypeptides whose expression is decreased in a patient having a bipolar disorder, agonists would be particularly useful in the methods of the invention. For those nucleic acid molecules or polypeptides whose expression is increased in a patient having a bipolar disorder (e.g., those identified in Table 4), antagonists would be particularly useful in the methods of the invention.

Each of the DNA sequences listed herein may also be used in the discovery and development of a therapeutic compound for the treatment of bipolar disorder. The encoded protein, upon expression, can be used as a target for the screening of drugs. Additionally, the DNA sequences encoding the amino terminal regions of the encoded protein or Shine-Delgarno or other translation facilitating sequences of the respective mRNA can be used to construct sequences that promote the expression of the coding sequence of interest. Such sequences may be isolated by standard techniques (Ausubel et al., supra).

Optionally, compounds identified in any of the above-described assays may be confirmed as useful in an assay for compounds that modulate the activity of a mitochondrial or proteasomal pathway.

Small molecules of the invention preferably have a molecular weight below 2,000 daltons, more preferably between 300 and 1,000 daltons, and most preferably between 400 and 700 daltons. It is preferred that these small molecules are organic molecules.

Test Compounds and Extracts

In general, compounds capable of increasing the activity of a mitochondrial energy or proteasomal pathway are identified from large libraries of both natural product or synthetic (or semi-synthetic) extracts or chemical libraries or from polypeptide or nucleic acid libraries (e.g., Table 2 or Table 4), according to methods known in the art. Those skilled in the field of drug discovery and development will understand that the precise source of test extracts or compounds is not critical to the screening procedure(s) of the invention. Compounds used in screens may include known compounds (for example, known therapeutics used for other diseases or disorders). Alternatively, virtually any number of unknown chemical extracts or compounds can be screened using the methods described herein. Examples of such extracts or compounds include, but are not limited to, plant-, fungal-, prokaryotic- or animal-based extracts, fermentation broths, and synthetic compounds, as well as modification of existing compounds. Numerous methods are also available for generating random or directed synthesis (e.g., semi-synthesis or total synthesis) of any number of chemical compounds, including, but not limited to, saccharide-, lipid-, peptide-, and nucleic acid-based compounds. Synthetic compound libraries are commercially available from Brandon Associates (Merrimack, N.H.) and Aldrich Chemical (Milwaukee, Wis.). Alternatively, libraries of natural compounds in the form of bacterial, fungal, plant, and animal extracts are commercially available from a number of sources, including Biotics (Sussex, UK), Xenova (Slough, UK), Harbor Branch Oceangraphics Institute (Ft. Pierce, Fla.), and PharmaMar, U.S.A. (Cambridge, Mass.). In addition, natural and synthetically produced libraries are produced, if desired, according to methods known in the art, e.g., by standard extraction and fractionation methods. Furthermore, if desired, any library or compound is readily modified using standard chemical, physical, or biochemical methods.

In addition, those skilled in the art of drug discovery and development readily understand that methods for dereplication (e.g., taxonomic dereplication, biological dereplication, and chemical dereplication, or any combination thereof) or the elimination of replicates or repeats of materials already known for their activity should be employed whenever possible.

When a crude extract is found to increase the activity of a mitochondrial energy metabolism or proteasomal pathway, or to binding a component of such a pathway, further fractionation of the positive lead extract is necessary to isolate chemical constituents responsible for the observed effect. Thus, the goal of the extraction, fractionation, and purification process is the careful characterization and identification of a chemical entity within the crude extract that increases the activity of a mitochondrial energy metabolism or proteasomal pathway. Methods of fractionation and purification of such heterogenous extracts are known in the art. If desired, compounds shown to be useful as therapeutics for the treatment of a human bipolar disorder are chemically modified according to methods known in the art.

Treatment of a Bipolar Disorder

Gene therapy is another therapeutic approach for preventing or ameliorating a bipolar disorder caused by the inadequate expression of a mitochondrial energy metabolism gene, proteasomal gene, or other nucleic acid molecule listed in Table 2 or Table 4. Such nucleic acid molecules can be delivered to cells that lack sufficient, normal protein expression or biological activity. The nucleic acid molecules must be delivered to those cells in a form in which they can be taken up by the cells and so that sufficient levels of protein can be produced to increase, for example, mitochondrial energy metabolism or proteasomal function in a patient having a bipolar disorder.

Transducing viral (e.g., retroviral, adenoviral, and adeno-associated viral) vectors can be used for somatic cell gene therapy, especially because of their high efficiency of infection and stable integration and expression (see, e.g., Cayouette et al., Human Gene Therapy 8:423-430, 1997; Kido et al., Current Eye Research 15:833-844, 1996; Bloomer et al., Journal of Virology 71:6641-6649, 1997; Naldini et al., Science 272:263-267, 1996; and Miyoshi et al., Proc. Natl. Acad. Sci. U.S.A. 94:10319, 1997). For example, a full length gene (e.g., a nucleic acid molecule listed in Table 2 or Table 4), or a portion thereof, can be cloned into a retroviral vector and expression can be driven from its endogenous promoter, from the retroviral long terminal repeat, or from a promoter specific for a target cell type of interest (e.g., a cell of the central nervous system). Other viral vectors that can be used include, for example, a vaccinia virus, a bovine papilloma virus, or a herpes virus, such as Epstein-Barr Virus (also see, for example, the vectors of Miller, Human Gene Therapy 15-14, 1990; Friedman, Science 244:1275-1281, 1989; Eglitis et al., BioTechniques 6:608-614, 1988; Tolstoshev et al., Current Opinion in Biotechnology 1:55-61, 1990; Sharp, The Lancet 337:1277-1278, 1991; Cornetta et al., Nucleic Acid Research and Molecular Biology 36:311-322, 1987; Anderson, Science 226:401-409, 1984; Moen, Blood Cells 17:407-416, 1991; Miller et al., Biotechnology 7:980-990, 1989; Le Gal La Salle et al., Science 259:988-990, 1993; and Johnson, Chest 107:77S-83S, 1995). Retroviral vectors are particularly well developed and have been used in clinical settings (Rosenberg et al., N. Engl. J. Med 323:370, 1990; Anderson et al., U.S. Pat. No. 5,399,346). Most preferably, a viral vector is used to administer the gene of interest (e.g., nucleic acids listed in Table 2 or Table 4) systemically or to a cell of the central nervous system, such as a neuron or glial cell. Methods for introducing therapeutic nucleic acids to a cell of the nervous system are described in U.S. Pat. No. 6,503,888.

Non-viral approaches can also be employed for the introduction of therapeutic to a cell of a patient having a bipolar disorder. For example, a nucleic acid molecule can be introduced into a cell by administering the nucleic acid in the presence of lipofection (Feigner et al., Proc. Natl. Acad. Sci. U.S.A. 84:7413, 1987; Ono et al., Neuroscience Letters 17:259, 1990; Brigham et al., Am. J. Med. Sci. 298:278, 1989; Staubinger et al., Methods in Enzymology 101:512, 1983), asialoorosomucoid-polylysine conjugation (Wu et al., Journal of Biological Chemistry 263:14621, 1988; Wu et al., Journal of Biological Chemistry 264:16985, 1989), or by micro-injection under surgical conditions (Wolff et al., Science 247:1465, 1990). Preferably the nucleic acids are administered in combination with a liposome and protamine.

Gene transfer can also be achieved using non-viral means involving transfection in vitro. Such methods include the use of calcium phosphate, DEAE dextran, electroporation, and protoplast fusion. Liposomes can also be potentially beneficial for delivery of DNA into a cell. Transplantation of normal genes into the affected tissues of a patient can also be accomplished by transferring a normal nucleic acid into a cultivatable cell type ex vivo (e.g., an autologous or heterologous primary cell or progeny thereof), after which the cell (or its descendants) are injected into a targeted tissue.

cDNA expression for use in gene therapy methods can be directed from any suitable promoter (e.g., the human cytomegalovirus (CMV), simian virus 40 (SV40), or metallothionein promoters), and regulated by any appropriate mammalian regulatory element. For example, if desired, enhancers known to preferentially direct gene expression in specific cell types, such as cells of the central nervous system or their associated glial cells, can be used to direct the expression of a nucleic acid. The enhancers used can include, without limitation, those that are characterized as tissue- or cell-specific enhancers. Alternatively, if a genomic clone is used as a therapeutic construct, regulation can be mediated by the cognate regulatory sequences or, if desired, by regulatory sequences derived from a heterologous source, including any of the promoters or regulatory elements described above.

Another therapeutic approach included in the invention involves administration of a recombinant therapeutic, such as a recombinant nuclear encoded mitochondrial metabolism or proteasomal polypeptide, either directly to the site of a potential or actual disease-affected tissue (for example, by stereotaxic injection into the ventricles of the brain or into the cerebrospinal fluid) or systemically (for example, by any conventional recombinant protein administration technique). The dosage of the administered protein depends on a number of factors, including the size and health of the individual patient. For any particular subject, the specific dosage regimes should be adjusted over time according to the individual need and the professional judgement of the person administering or supervising the administration of the compositions. Generally, between 0.1 mg and 100 mg, is administered per day to an adult in any pharmaceutically acceptable formulation.

Pharmaceutical Therapeutics

The invention provides a simple means for identifying compositions (including nucleic acids, peptides, small molecule inhibitors, and mimetics) capable of acting as therapeutics for the treatment of a bipolar disorder. Accordingly, a chemical entity discovered to have medicinal value using the methods described herein is useful as a drug or as information for structural modification of existing compounds, e.g., by rational drug design. Such methods are useful for screening compounds having an effect on a variety of mental conditions characterized by a decrease in the expression of a mitochondrial energy metabolism or proteasomal gene.

For therapeutic uses, the compositions or agents identified using the methods disclosed herein may be administered systemically, for example, formulated in a pharmaceutically-acceptable buffer such as physiological saline. Preferable routes of administration include, for example, subcutaneous, intravenous, interperitoneally, intramuscular, or intradermal injections that provide continuous, sustained levels of the drug in the patient. Treatment of human patients or other animals will be carried out using a therapeutically effective amount of a bipolar disorder therapeutic in a physiologically-acceptable carrier. Suitable carriers and their formulation are described, for example, in Remington's Pharmaceutical Sciences by E. W. Martin. The amount of the therapeutic agent to be administered varies depending upon the manner of administration, the age and body weight of the patient, and with the clinical symptoms of the bipolar disorder (e.g., mania, depression, mixed, or rapid cycling). Generally, amounts will be in the range of those used for other agents used in the treatment of other diseases associated with a bipolar disorder, although in certain instances lower amounts will be needed because of the increased specificity of the compound. A compound is administered at a dosage that controls the clinical or physiological symptoms of a bipolar disorder as determined by psychiatric evaluation or by a diagnostic method of the invention that assays the expression of a nucleic acid molecule listed in Table 2 or Table 4, or the biological activity of a polypeptide encoded by such a nucleic acid molecule.

Formulation of Pharmaceutical Compositions

The administration of a compound for the treatment of a bipolar disorder may be by any suitable means that results in a concentration of the therapeutic that, combined with other components, is effective in ameliorating, reducing, or stabilizing a bipolar disorder. The compound may be contained in any appropriate amount in any suitable carrier substance, and is generally present in an amount of 1-95% by weight of the total weight of the composition. The composition may be provided in a dosage form that is suitable for parenteral (e.g., subcutaneously, intravenously, intramuscularly, or intraperitoneally) administration route. The pharmaceutical compositions may be formulated according to conventional pharmaceutical practice (see, e.g., Remington: The Science and Practice of Pharmacy (20th ed.), ed. A. R. Gennaro, Lippincott Williams & Wilkins, 2000 and Encyclopedia of Pharmaceutical Technology, eds. J. Swarbrick and J. C. Boylan, 1988-1999, Marcel Dekker, New York).

Pharmaceutical compositions according to the invention may be formulated to release the active compound substantially immediately upon administration or at any predetermined time or time period after administration. The latter types of compositions are generally known as controlled release formulations, which include (i) formulations that create a substantially constant concentration of the drug within the body over an extended period of time; (ii) formulations that after a predetermined lag time create a substantially constant concentration of the drug within the body over an extended period of time; (iii) formulations that sustain action during a predetermined time period by maintaining a relatively, constant, effective level in the body with concomitant minimization of undesirable side effects associated with fluctuations in the plasma level of the active substance (sawtooth kinetic pattern); (iv) formulations that localize action by, e.g., spatial placement of a controlled release composition adjacent to or in the central nervous system or cerebrospinal fluid; (v) formulations that allow for convenient dosing, such that doses are administered, for example, once every one or two weeks; and (vi) formulations that target a bipolar disorder by using carriers or chemical derivatives to deliver the therapeutic agent to a particular cell type or cell organelle (e.g., mitochondria or proteasome) whose function is perturbed in a bipolar disorder. For some applications, controlled release formulations obviate the need for frequent dosing during the day to sustain the plasma level at a therapeutic level.

Any of a number of strategies can be pursued in order to obtain controlled release in which the rate of release outweighs the rate of metabolism of the compound in question. In one example, controlled release is obtained by appropriate selection of various formulation parameters and ingredients, including, e.g., various types of controlled release compositions and coatings. Thus, the bipolar disorder therapeutic is formulated with appropriate excipients into a pharmaceutical composition that, upon administration, releases the bipolar disorder therapeutic in a controlled manner. Examples include single or multiple unit tablet or capsule compositions, oil solutions, suspensions, emulsions, microcapsules, microspheres, molecular complexes, nanoparticles, patches, and liposomes.

Parenteral Compositions

The pharmaceutical composition may be administered parenterally by injection, infusion or implantation (subcutaneous, intravenous, intramuscular, intraperitoneal, or the like) in dosage forms, formulations, or via suitable delivery devices or implants containing conventional, non-toxic pharmaceutically acceptable carriers and adjuvants. The formulation and preparation of such compositions are well known to those skilled in the art of pharmaceutical formulation. Formulations can be found in Remington: The Science and Practice of Pharmacy, supra.

Compositions for parenteral use may be provided in unit dosage forms (e.g., in single-dose ampoules), or in vials containing several doses and in which a suitable preservative may be added (see below). The composition may be in form of a solution, a suspension, an emulsion, an infusion device, or a delivery device for implantation, or it may be presented as a dry powder to be reconstituted with water or another suitable vehicle before use. Apart from the active bipolar disorder therapeutic (s), the composition may include suitable parenterally acceptable carriers and/or excipients. The active bipolar disorder therapeutic (s) may be incorporated into microspheres, microcapsules, nanoparticles, liposomes, or the like for controlled release. Furthermore, the composition may include suspending, solubilizing, stabilizing, pH-adjusting agents, tonicity adjusting agents, and/or dispersing, agents.

As indicated above, the pharmaceutical compositions according to the invention may be in the form suitable for sterile injection. To prepare such a composition, the suitable active bipolar disorder therapeutic(s) are dissolved or suspended in a parenterally acceptable liquid vehicle. Among acceptable vehicles and solvents that may be employed are water, water adjusted to a suitable pH by addition of an appropriate amount of hydrochloric acid, sodium hydroxide or a suitable buffer, 1,3-butanediol, Ringer's solution, and isotonic sodium chloride solution and dextrose solution. The aqueous formulation may also contain one or more preservatives (e.g., methyl, ethyl or n-propyl p-hydroxybenzoate). In cases where one of the compounds is only sparingly or slightly soluble in water, a dissolution enhancing or solubilizing agent can be added, or the solvent may include 10-60% w/w of propylene glycol or the like.

Controlled Release Parenteral Compositions

Controlled release parenteral compositions may be in form of aqueous suspensions, microspheres, microcapsules, magnetic microspheres, oil solutions, oil suspensions, or emulsions. Alternatively, the active drug may be incorporated in biocompatible carriers, liposomes, nanoparticles, implants, or infusion devices.

Materials for use in the preparation of microspheres and/or microcapsules are, e.g., biodegradable/bioerodible polymers such as polygalactin, poly-(isobutyl cyanoacrylate), poly(2-hydroxyethyl-L-glutamnine) and, poly(lactic acid). Biocompatible carriers that may be used when formulating a controlled release parenteral formulation are carbohydrates (e.g., dextrans), proteins (e.g., albumin), lipoproteins, or antibodies. Materials for use in implants can be non-biodegradable (e.g., polydimethyl siloxane) or biodegradable (e.g., poly(caprolactone), poly(lactic acid), poly(glycolic acid) or poly(ortho esters) or combinations thereof).

Solid Dosage Forms For Oral Use

Formulations for oral use include tablets containing the active ingredient(s) in a mixture with non-toxic pharmaceutically acceptable excipients. Such formulations are known to the skilled artisan. Excipients may be, for example, inert diluents or fillers (e.g., sucrose, sorbitol, sugar, mannitol, microcrystalline cellulose, starches including potato starch, calcium carbonate, sodium chloride, lactose, calcium phosphate, calcium sulfate, or sodium phosphate); granulating and disintegrating agents (e.g., cellulose derivatives including microcrystalline cellulose, starches including potato starch, croscarmellose sodium, alginates, or alginic acid); binding agents (e.g., sucrose, glucose, sorbitol, acacia, alginic acid, sodium alginate, gelatin, starch, pregelatinized starch, microcrystalline cellulose, magnesium aluminum silicate, carboxymethylcellulose sodium, methylcellulose, hydroxypropyl methylcellulose, ethylcellulose, polyvinylpyrrolidone, or polyethylene glycol); and lubricating agents, glidants, and antiadhesives (e.g., magnesium stearate, zinc stearate, stearic acid, silicas, hydrogenated vegetable oils, or talc). Other pharmaceutically acceptable excipients can be colorants, flavoring agents, plasticizers, humectants, buffering agents, and the like.

The tablets may be uncoated or they may be coated by known techniques, optionally to delay disintegration and absorption in the gastrointestinal tract and thereby providing a sustained action over a longer period. The coating may be adapted to release the active drug in a predetermined pattern (e.g., in order to achieve a controlled release formulation) or it may be adapted not to release the active drug until after passage of the stomach (enteric coating). The coating may be a sugar coating, a film coating (e.g., based on hydroxypropyl methylcellulose, methylcellulose, methyl hydroxyethylcellulose, hydroxypropylcellulose, carboxymethylcellulose, acrylate copolymers, polyethylene glycols and/or polyvinylpyrrolidone), or an enteric coating (e.g., based on methacrylic acid copolymer, cellulose acetate phthalate, hydroxypropyl methylcellulose phthalate, hydroxypropyl methylcellulose acetate succinate, polyvinyl acetate phthalate, shellac, and/or ethylcellulose). Furthermore, a time delay material such as, e.g., glyceryl monostearate or glyceryl distearate may be employed.

The solid tablet compositions may include a coating adapted to protect the composition from unwanted chemical changes, (e.g., chemical degradation prior to the release of the active bipolar disorder therapeutic substance). The coating may be applied on the solid dosage form in a similar manner as that described in Encyclopedia of Pharmaceutical Technology, supra.

At least two bipolar disorder therapeutics may be mixed together in the tablet, or may be partitioned. In one example, the first bipolar disorder therapeutic is contained on the inside of the tablet, and the second bipolar disorder therapeutic is on the outside, such that a substantial portion of the second bipolar disorder therapeutic is released prior to the release of the first bipolar disorder therapeutic.

Formulations for oral use may also be presented as chewable tablets, or as hard gelatin capsules wherein the active ingredient is mixed with an inert solid diluent (e.g., potato starch, lactose, microcrystalline cellulose, calcium carbonate, calcium phosphate or kaolin), or as soft gelatin capsules wherein the active ingredient is mixed with water or an oil medium, for example, peanut oil, liquid paraffin, or olive oil. Powders and granulates may be prepared using the ingredients mentioned above under tablets and capsules in a conventional manner using, e.g., a mixer, a fluid bed apparatus or a spray drying equipment.

Controlled Release Oral Dosage Forms

Controlled release compositions for oral use may, e.g., be constructed to release the active bipolar disorder therapeutic by controlling the dissolution and/or the diffusion of the active substance.

Dissolution or diffusion controlled release can be achieved by appropriate coating of a tablet, capsule, pellet, or granulate formulation of compounds, or by incorporating the compound into an appropriate matrix. A controlled release coating may include one or more of the coating substances mentioned above and/or, e.g., shellac, beeswax, glycowax, castor wax, carnauba wax, stearyl alcohol, glyceryl monostearate, glyceryl distearate, glycerol palmitostearate, ethylcellulose, acrylic resins, dl-polylactic acid, cellulose acetate butyrate, polyvinyl chloride, polyvinyl acetate, vinyl pyrrolidone, polyethylene, polymethacrylate, methylmethacrylate, 2-hydroxymethacrylate, methacrylate hydrogels, 1,3 butylene glycol, ethylene glycol methacrylate, and/or polyethylene glycols. In a controlled release matrix formulation, the matrix material may also include, e.g., hydrated metylcellulose, carnauba wax and stearyl alcohol, carbopol 934, silicone, glyceryl tristearate, methyl acrylate-methyl methacrylate, polyvinyl chloride, polyethylene, and/or halogenated fluorocarbon.

A controlled release composition containing one or more therapeutic compounds may also be in the form of a buoyant tablet or capsule (i.e., a tablet or capsule that, upon oral administration, floats on top of the gastric content for a certain period of time). A buoyant tablet formulation of the compound(s) can be prepared by granulating a mixture of the compound(s) with excipients and 20-75% w/w of hydrocolloids, such as hydroxyethylcellulose, hydroxypropylcellulose, or hydroxypropylmethylcellulose. The obtained granules can then be compressed into tablets. On contact with the gastric juice, the tablet forms a substantially water-impermeable gel barrier around its surface. This gel barrier takes part in maintaining a density of less than one, thereby allowing the tablet to remain buoyant in the gastric juice.

Combination Therapies

Optionally, a bipolar disorder therapeutic may be administered in combination with any other standard bipolar disorder therapy; such methods are known to the skilled artisan and described in Remington's Pharmaceutical Sciences by E. W. Martin. A bipolar disorder therapeutic of the invention may be administered in combination with mood stabilizing drugs, such as lithium and carbamazepine, anticonvulsant medications, such as valproate, lamotrigine, gabapentin, and topiramate, atypical antipsychotic drugs, such as clozapine or olanzapine, or for particularly severe cases, in combination with electroconvulsive therapy.

Patient Monitoring

The disease state or treatment of a patient having a bipolar disorder can be monitored using the methods and compositions of the invention. Some embodiments, a microarray or quantitative real-time PCR is used to assay the expression profile of at least one of the nucleic acids listed in Table 2 or Table 4. Such monitoring may be useful, for example, in assessing the efficacy of a particular drug in a patient or in assessing patient compliance with a treatment regimen. Therapeutics that increase the expression of at least one nuclear encoded mitochondrial energy metabolism or proteasomal nucleic acid molecule or polypeptide (e.g., at least one or more of the nucleic acids listed in Table 2) are taken as particularly useful in the invention.

Other Embodiments

From the foregoing description, it will be apparent that variations and modifications may be made to the invention described herein to adopt it to various usages and conditions. Such embodiments are also within the scope of the following claims. All patents and publications mentioned in this specification are herein incorporated by reference to the same extent as if each independent patent and publication was specifically and individually indicated to be incorporated by reference.

What is claimed is:

1. A microarray comprising at least two nuclear encoded mitochondrial energy metabolism nucleic acid molecules, or fragments thereof, bound to a solid support, wherein at least 90% of the nucleic acid molecules on said support are mitochondrial energy metabolism nucleic acid molecules.

2. The microarray of claim 1, wherein said array comprises nuclear encoded mitochondrial energy metabolism nucleic acid molecules, or fragments thereof, selected from the group consisting of an ATP synthase (mitochondrial F0 complex, subunit c, isoform 3), VDAC 1 pseudogene (porin protein, isoform 1), ubiquinone-binding protein, ATP synthase (mitochondrial F0 complex, subunit d), mitochondrial ribosomal protein L3, cytochrome c oxidase subunit VIIb, ATP synthase (mitochondrial F0 complex, subunit f, isoform 2), dynamin 1-like protein, voltage-dependent anion channel 2 (porin), Cytochrome c oxidase subunit VIIa polypeptide 2 (liver), ATP synthase (mitochondrial F1 complex, O subunit), voltage-dependent anion channel 1 (porin), single-stranded DNA binding protein, fumarate hydratase, solute carrier family 25 (member 4), ATP synthase (mitochondrial F1 complex gamma polypeptide 1), NADH dehydrogenase ((ubiquinone) 1 alpha/beta subcomplex 1, 8 kDa), and 3-oxoacid CoA transferase nucleic acid molecules.

3. A microarray comprising at least two nuclear encoded mitochondrial energy metabolism polypeptides, or fragments thereof, bound to a solid support, wherein at least 90% of the polypeptides on said support are nuclear encoded mitochondrial energy metabolism polypeptides.

4. A method of diagnosing a patient having, or having a propensity to develop, a bipolar disorder, said method comprising determining the level of expression of a nuclear encoded mitochondrial energy metabolism nucleic acid molecule in a patient sample, wherein a decreased level of expression relative to the level of expression in a control sample, indicates that said patient has or has a propensity to develop a bipolar disorder.

5. The method of claim 4, wherein said patient sample is a blood sample.

6. A method of diagnosing a patient having, or having a propensity to develop, a bipolar disorder, said method comprising determining the level of expression of a nuclear encoded mitochondrial energy metabolism polypeptide in a patient sample, wherein a decreased level of expression relative to the level of expression in a control sample, indicates that said patient has or has a propensity to develop a bipolar disorder.

7. The method of claim 6, wherein said patient sample is a blood sample.

8. A method of monitoring a patient having a bipolar disorder, said method comprising determining the level of expression of a nuclear encoded mitochondrial energy metabolism nucleic acid or polypeptide in a patient sample, wherein an alteration in the level of expression relative to the level of expression in a control sample indicates the severity of bipolar disorder in said patient.

9. The method of claim 8, wherein said patient sample is a blood sample.

10. A method of identifying a candidate compound that ameliorates a bipolar disorder, said method comprising contacting a cell that expresses a nuclear encoded mitochondrial energy metabolism nucleic acid molecule with a candidate compound, and comparing the level of expression of said nucleic acid molecule in said cell contacted by said candidate compound with the level of expression in a control cell not contacted by said candidate compound, wherein an increase in expression of said nuclear encoded mitochondrial energy metabolism nucleic acid molecule identifies said candidate compound as a candidate compound that ameliorates a bipolar disorder.

11. A method of identifying a candidate compound that ameliorates a bipolar disorder, the method comprising contacting a cell that expresses a nuclear encoded mitochondrial energy metabolism polypeptide with a candidate compound, and comparing the level of expression of said polypeptide in said cell contacted by said candidate compound with the level of polypeptide expression in a control cell not contacted by said candidate compound, wherein an increase in the expression of said nuclear encoded mitochondrial energy metabolism polypeptide identifies said candidate compound as a candidate compound that ameliorates a bipolar disorder.

12. A method of identifying a candidate compound that ameliorates a bipolar disorder, the method comprising contacting a cell that expresses a nuclear encoded mitochondrial energy metabolism polypeptide with a candidate compound, and comparing the biological activity of said polypeptide in said cell contacted by said candidate compound with the level of biological activity in a control cell not contacted by said candidate compound, wherein an increase in the biological activity of said nuclear encoded mitochondrial energy metabolism polypeptide identifies said candidate compound as a candidate compound that ameliorates a bipolar disorder.

13. A method of identifying a candidate compound that ameliorates a bipolar disorder, the method comprising
a) contacting a cell with a candidate compound;
b) obtaining a nucleic acid from said cell;
c) contacting a microarray of claim 1 with said nucleic acid; and
d) detecting an increase in expression level of a nuclear encoded mitochondrial energy metabolism nucleic acid molecule in said cell contacted with said candidate compound compared to a control cell, wherein said increase identifies the candidate compound as a candidate compound that ameliorates a bipolar disorder.

14. A method of identifying a candidate compound that ameliorates a bipolar disorder, said method comprising the steps of
a) contacting a microarray of claim 3 with a candidate compound; and
b) detecting binding of said candidate compound to a nuclear encoded mitochondrial energy metabolism polypeptide, wherein said binding identifies the compound as a candidate compound that ameliorates a bipolar disorder.

15. A microarray comprising at least two nucleic acid molecules, or fragments thereof, bound to a solid support, wherein at least 90% of the nucleic acids molecules on the support encode a proteasomal polypeptide.

16. The microarray of claim 15, wherein said nucleic acid molecules, or fragments thereof, are selected from the group consisting of a Sec61 gamma, protein-L-isoaspartate (D-aspartate) O-methyltransferase, F-box only protein 9, and proteasome-subunit z nucleic acid molecule.

17. A microarray comprising at least two proteasomal polypeptides, or fragments thereof, bound to a solid support, wherein at least 90% of the polypeptides on the support are proteasomal polypeptides.

18. A method of diagnosing a patient having, or having a propensity to develop, a bipolar disorder, said method comprising determining the level of expression of a nucleic acid that encodes a proteasomal polypeptide in a patient sample, wherein a decreased level of expression relative to the level of expression in a control sample, indicates that said patient has or has a propensity to develop a bipolar disorder.

19. The method of claim 18, wherein said patient sample is a blood sample.

20. A method of diagnosing a patient having, or having a propensity to develop, a bipolar disorder, said method comprising determining the level of expression of a proteasomal polypeptide in a patient sample, wherein a decreased level of expression relative to the level of expression in a control sample, indicates that said patient has or has a propensity to develop a bipolar disorder.

21. The method of claim 20, wherein said patient sample is a blood sample.

22. A method of monitoring a patient having a bipolar disorder, said method comprising determining the level of expression of a proteasomal nucleic acid molecule or polypeptide in a patient sample, wherein an alteration in the level of expression relative to the level of expression in a control sample indicates the severity of a bipolar disorder in said patient.

23. A method of identifying a candidate compound that ameliorates a bipolar disorder, said method comprising contacting a cell that expresses a nucleic acid molecule encoding a proteasomal polypeptide with a candidate compound, and comparing the level of expression of said nucleic acid molecule in said cell contacted by said candidate compound with the level of expression in a control cell not contacted by said candidate compound, wherein an increase in expression of said nucleic acid molecule encoding a proteasomal polypeptide identifies said candidate compound as a candidate compound that ameliorates a bipolar disorder.

24. A method of identifying a candidate compound that ameliorates a bipolar disorder, the method comprising contacting a cell that expresses a proteasomal polypeptide with a candidate compound, and comparing the level of expression of said polypeptide in said cell contacted by said candidate compound with the level of polypeptide expression in a control cell not contacted by said candidate compound, wherein an increase in the expression of a proteasomal polypeptide identifies said candidate compound as a candidate compound that ameliorates a bipolar disorder.

25. A method of identifying a candidate compound that ameliorates a bipolar disorder, the method comprising
a) contacting a cell with a candidate compound;
b) obtaining a nucleic acid from said cell;
c) contacting a microarray of claim 15 with said nucleic acid; and
d) detecting an increase in expression level of a nucleic acid molecule encoding a proteasomal polypeptide in said cell contacted with said candidate compound compared to a control cell, wherein said increase identifies the candidate compound as a candidate compound that ameliorates a bipolar disorder.

26. A method of identifying a candidate compound that ameliorates a bipolar disorder, said method comprising the steps of
a) contacting a microarray of claim 17 with a candidate compound; and
b) detecting binding of said candidate compound to a proteasomal polypeptide, wherein said binding identifies the compound as a candidate compound that ameliorates a bipolar disorder.

27. A microarray comprising at least two nucleic acid molecules listed in Table 4, or fragments thereof, bound to a solid support, wherein at least 90% of the nucleic acids on said support are listed in Table 4.

28. A microarray comprising at least two polypeptides listed in Table 4, or fragments thereof, bound to a solid support, wherein at least 90% of the polypeptides on said support are polypeptides listed in Table 4.

29. A method of diagnosing a patient having, or having a propensity to develop, a bipolar disorder, said method comprising determining the level of expression of a nucleic acid listed in Table 4 in a patient sample, wherein an alteration in the level of expression relative to the level of expression in a control sample, indicates that said patient has or has a propensity to develop a bipolar disorder.

30. The method of claim 29, wherein said patient sample is a blood sample.

31. A method of diagnosing a patient having, or having a propensity to develop, a bipolar disorder, said method comprising determining the level of expression of a polypeptide encoded by a nucleic acid listed in Table 4 in a patient sample, wherein an altered level of expression relative to the level of expression in a control sample, indicates that said patient has or has a propensity to develop a bipolar disorder.

32. The method of claim 31, wherein said patient sample is a blood sample.

33. A method of monitoring a patient having a bipolar disorder, said method comprising determining the level of expression of a nucleic acid or polypeptide listed in Table 4 in a patient sample, wherein an alteration in the level of expression relative to the level of expression in a control sample indicates the severity of a bipolar disorder in said patient.

34. A method of identifying a candidate compound that ameliorates a bipolar disorder, said method comprising contacting a cell that expresses a nucleic acid molecule listed in Table 4 with a candidate compound, and comparing the level of expression of said nucleic acid molecule in said cell contacted by said candidate compound with the level of expression in a control cell not contacted by said candidate compound, wherein an alteration in expression of said nucleic acid molecule identifies said candidate compound as a candidate compound that ameliorates a bipolar disorder.

35. A method of identifying a candidate compound that ameliorates a bipolar disorder, the method comprising contacting a cell that expresses a polypeptide encoded by a nucleic acid molecule listed in Table 4 with a candidate compound, and comparing the level of expression of said polypeptide in said cell contacted by said candidate compound with the level of polypeptide expression in a control cell not contacted by said candidate compound, wherein an alteration in the expression of a polypeptide encoded by a nucleic acid molecule listed in Table 4 identifies said candidate compound as a candidate compound that ameliorates a bipolar disorder.

36. A method of identifying a candidate compound that ameliorates a bipolar disorder, the method comprising contacting a cell that expresses a polypeptide encoded by a nucleic acid molecule listed in Table 4 with a candidate compound, and comparing the biological activity of said polypeptide in said cell contacted by said candidate compound with the level of biological activity in a control cell not contacted by said candidate compound, wherein an alteration in the biological activity of said polypeptide identifies said candidate compound as a candidate compound that ameliorates a bipolar disorder.

37. A method of identifying a candidate compound that ameliorates a bipolar disorder, said method comprising
a) contacting a cell with a candidate compound;
b) obtaining a nucleic acid from said cell;
c) contacting a microarray of claim 27 with said nucleic acid; and
d) detecting an alteration in expression level of a nucleic acid molecule listed in Table 4 in said cell contacted with said candidate compound compared to a control cell, wherein said alteration identifies the candidate compound as a candidate compound that ameliorates a bipolar disorder.

38. A method of identifying a candidate compound that ameliorates a bipolar disorder, the method comprising the steps of
a) contacting a microarray of claim 28 with a candidate compound; and
b) detecting binding of said candidate compound to a proteasomal polypeptide, wherein said binding identifies the compound as a candidate compound that ameliorates a bipolar disorder.
